(19) United States
(12) Patent Application Publication
(10) Pub. No.: US 2024/0299187 A1
(43) Pub. Date: Sep. 12, 2024

(54) EXPANDABLE INTERBODY DEVICES AND INSTRUMENTS

(71) Applicant: Amplify Surgical, Inc., Irvine, CA (US)

(72) Inventors: Clark Hutton, Carlsbad, CA (US); Nathan Erickson, Beaverdam, UT (US)

(21) Appl. No.: 18/596,610

(22) Filed: Mar. 5, 2024

Related U.S. Application Data
(60) Provisional application No. 63/488,757, filed on Mar. 6, 2023.

(51) Int. Cl. A61F 2/44; A61F 2/30; A61F 2/46

(52) U.S. Cl. CPC A61F 2/447; A61F 2/4603; A61F 2002/30265; A61F 2002/30471; A61F 2002/30494; A61F 2002/30522; A61F 2002/3055; A61F 2002/30556; A61F 2002/30622; A61F 2002/30624; A61F 2002/4627

(57) ABSTRACT

The present technology is generally directed to expandable intervertebral devices, and associated devices, systems, and methods. In some embodiments, an intervertebral device is configured to transition between multiple different states, for example, between an unexpanded state and an expanded state. The expansion of the intervertebral device can include horizontal/lateral expansion and/or vertical/lordotic expansion. In some embodiments, the intervertebral device includes locking features configured to hold or “lock” the intervertebral device in an expanded state, e.g., to inhibit or prevent the intervertebral device from transitioning away from and/or out of the expanded state.

### CROSS-REFERENCE TO RELATED APPLICATION

The present application claims priority to U.S. Provisional Patent Application No. 63/488,757, filed Mar. 6, 2023, the disclosure of which is incorporated by reference herein in its entirety.

### TECHNICAL FIELD

This invention generally relates to the field of spinal surgery, and more particularly, to expandable interbody devices used in fusing adjacent vertebrae.

### BACKGROUND

In a vertebrate spine, the spinal disc and/or vertebral bodies may be displaced or damaged due to trauma, disease, degenerative defects, or wear over an extended period of time. One result of this displacement or damage to a spinal disc or vertebral body may be chronic back pain. A common procedure for treating damage or disease of the spinal disc or vertebral body may involve partial or complete removal of an intervertebral disc. An implant, which may be referred to as an interbody spacer, or intervertebral implant, can be inserted into the cavity created where the intervertebral disc was removed to help maintain height of the spine and/or restore stability to the spine. An interbody spacer may also provide a lordotic correction to the curvature of the spine. An example of an interbody spacer that has been commonly used is a fixed dimension cage, which typically is packed with bone and/or bone-growth-inducing materials.

One drawback of spacers known in the art is that they can be of fixed height and/or footprint and may not provide adequate or precise height restoration and support between affected vertebral bodies. Fixed size cages can also require more invasive procedures for implantation, due to their necessarily larger pre-implantation size. Accordingly, there is a need for an intervertebral implant which can be inserted along one axis and can be expanded to provide intervertebral support, lordotic correction, etc.

### BRIEF DESCRIPTION OF THE DRAWINGS

Exemplary embodiments of the invention will be best understood by reference to the drawings.

FIG. 1 is a side view of a spinal surgical system and a patient's spine, in accordance with embodiments of the disclosure.

FIG. 2A is a side view of an intervertebral device of in an unexpanded configuration and positioned within a vertebral body, in accordance with embodiments of the disclosure.

FIG. 2B is a side view of the intervertebral device of FIG. 2A in an expanded configuration.

FIG. 3A is a perspective view of an intervertebral device in a delivery state and configured in accordance with embodiments of the present technology; FIG. 3B is a back view of the intervertebral device of FIG. 3A; FIG. 3C is a front end view of the intervertebral device of FIG. 3A; FIG. 3D is a side view of the intervertebral device of FIG. 3A; FIG. 3E is a top view of the intervertebral device of FIG. 3A; and FIG. 3F is an exploded top view of the intervertebral device of FIG. 3A.

FIGS. 3G and 3H are a perspective view and a side view, respectively, of a first end body of the intervertebral device of FIG. 3A.

FIGS. 31 and 3J are a perspective view and a side view, respectively, of a second end body of the intervertebral device of FIG. 3A.

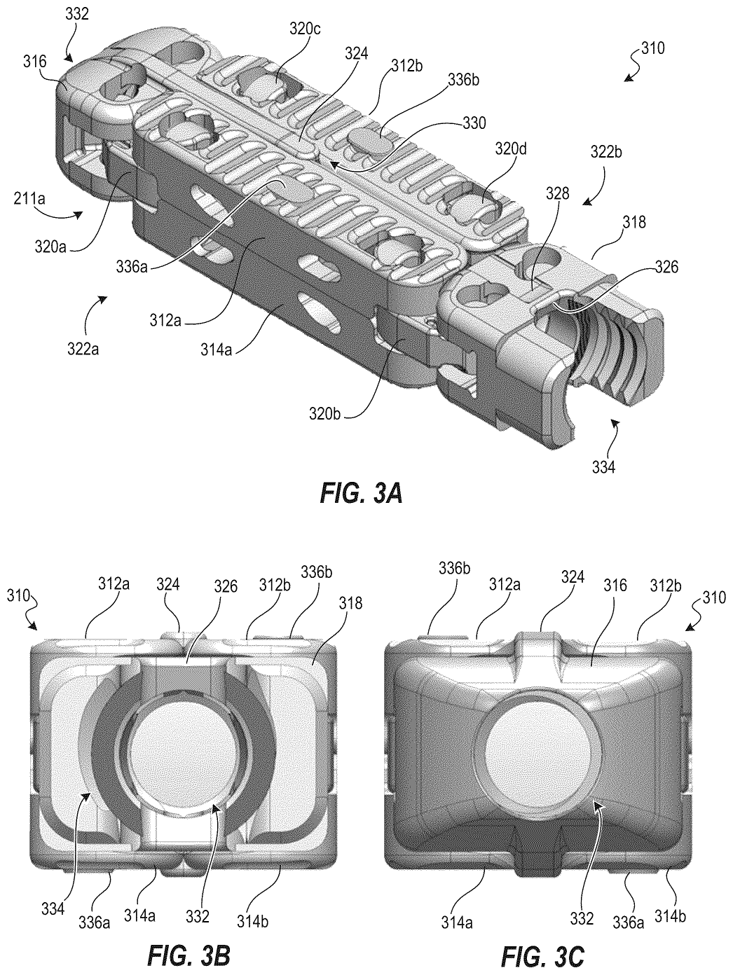

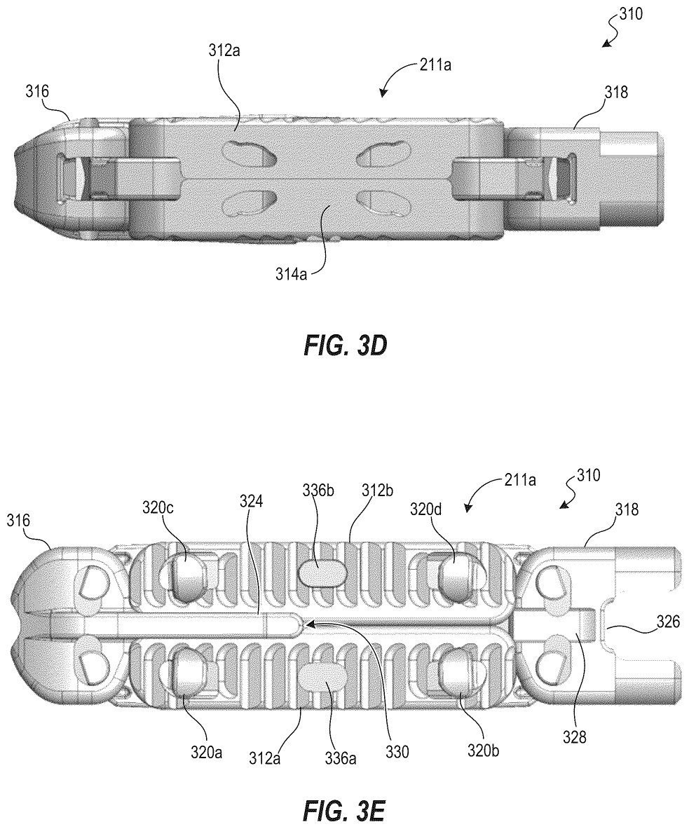

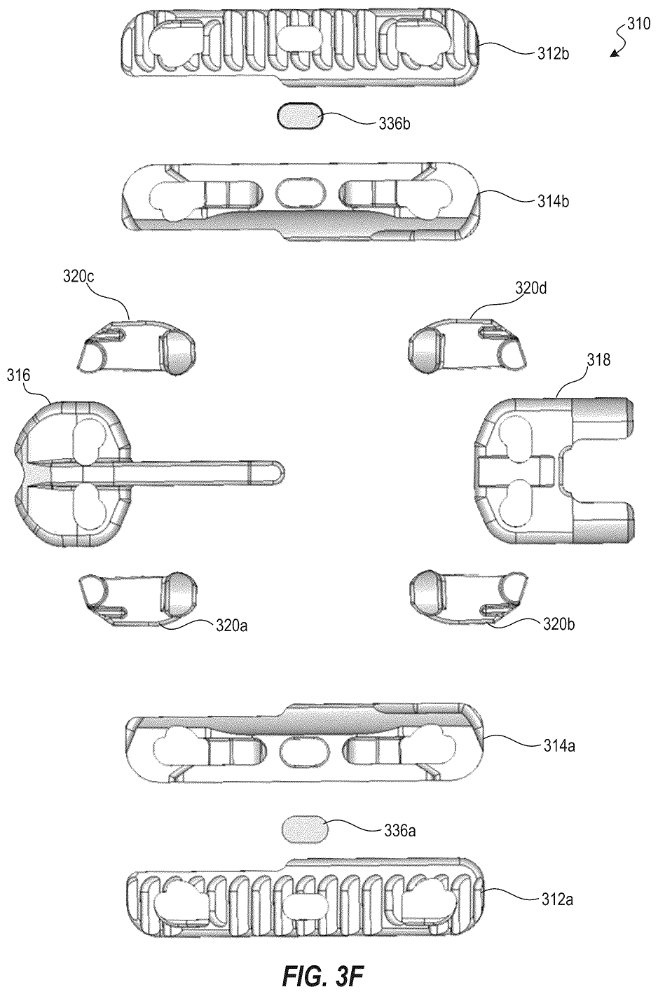

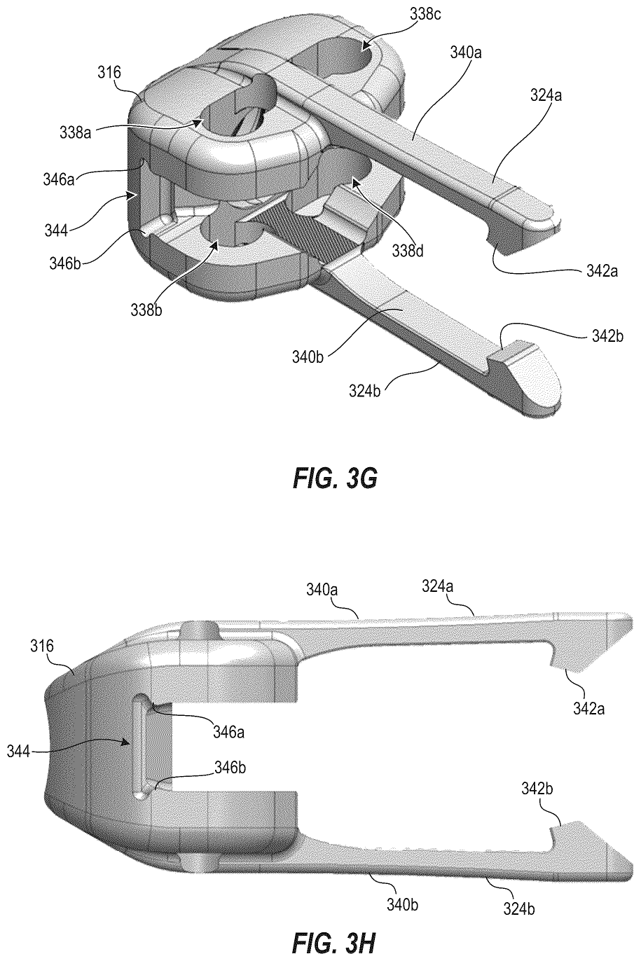

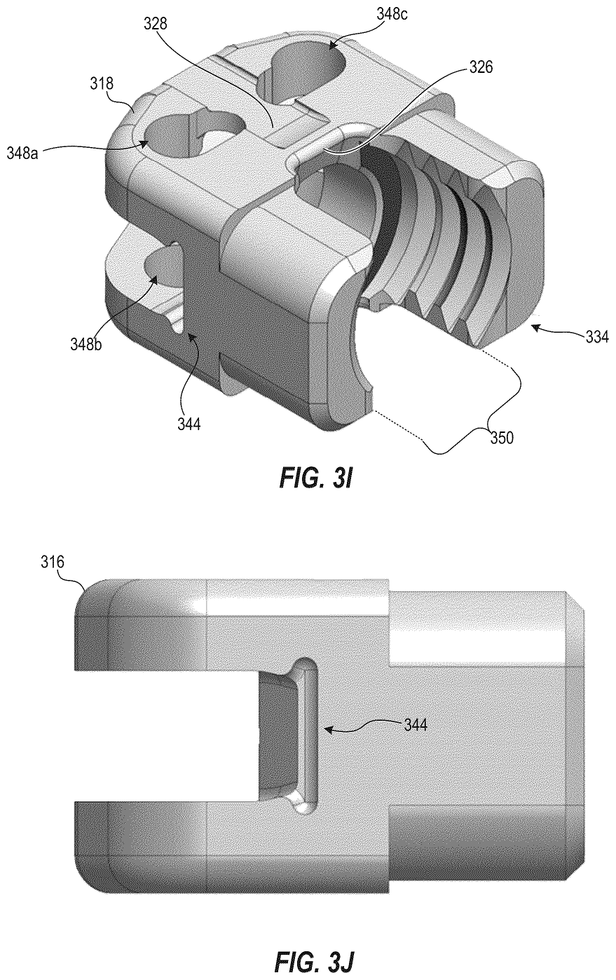

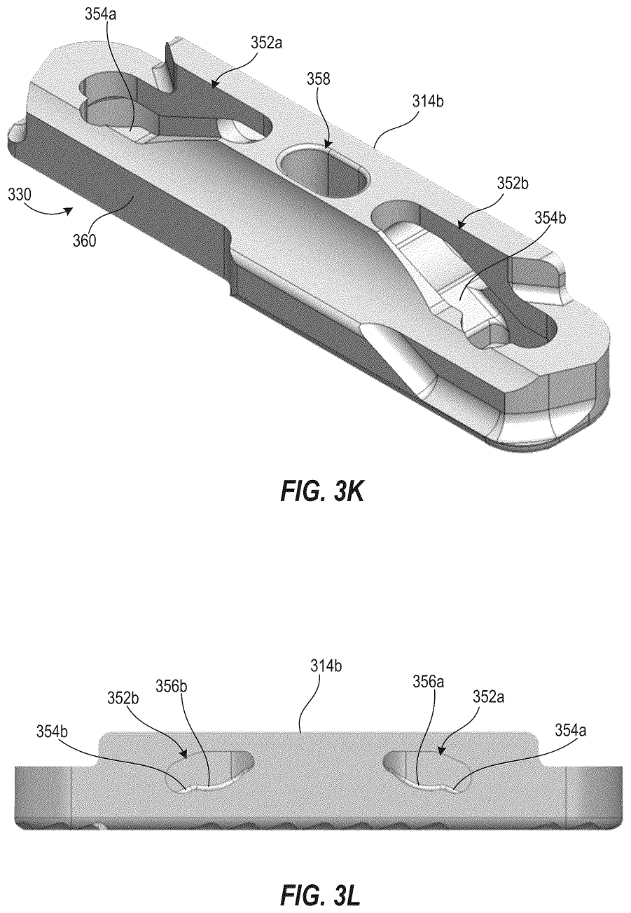

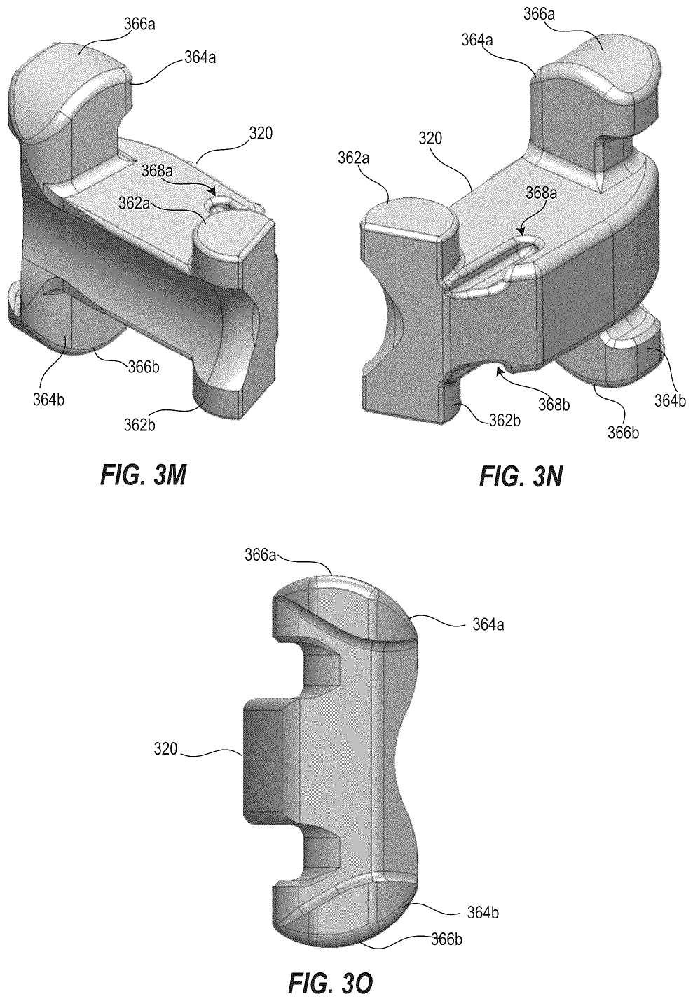

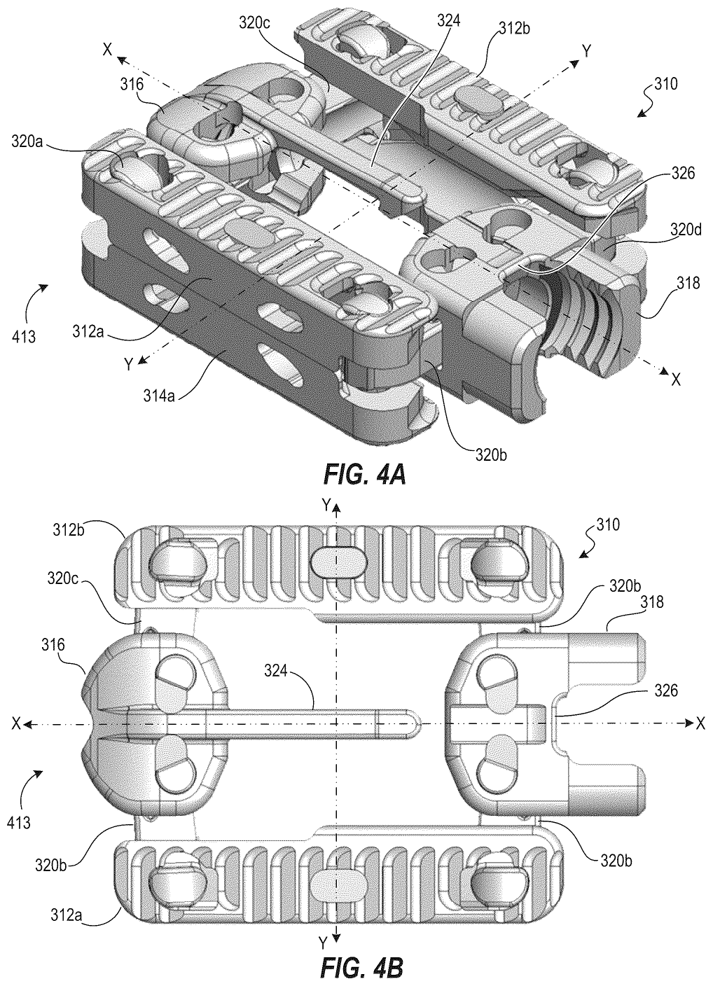

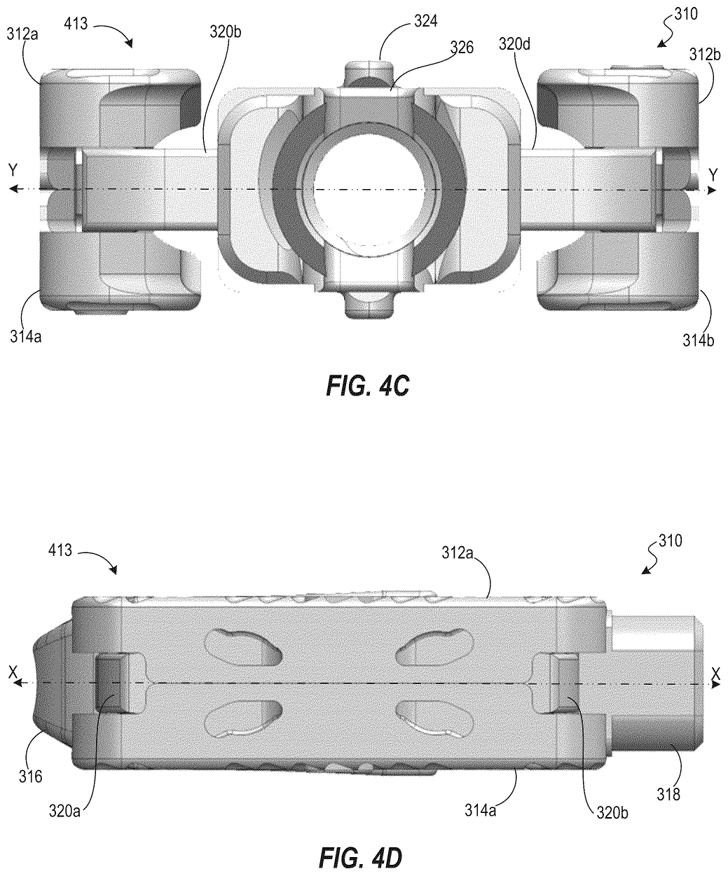

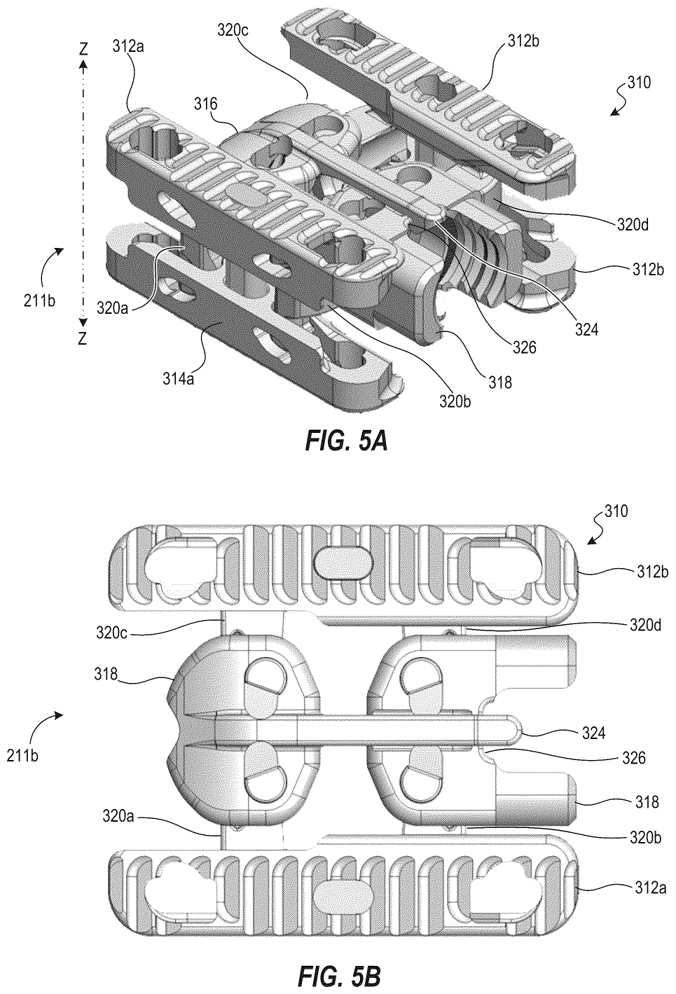

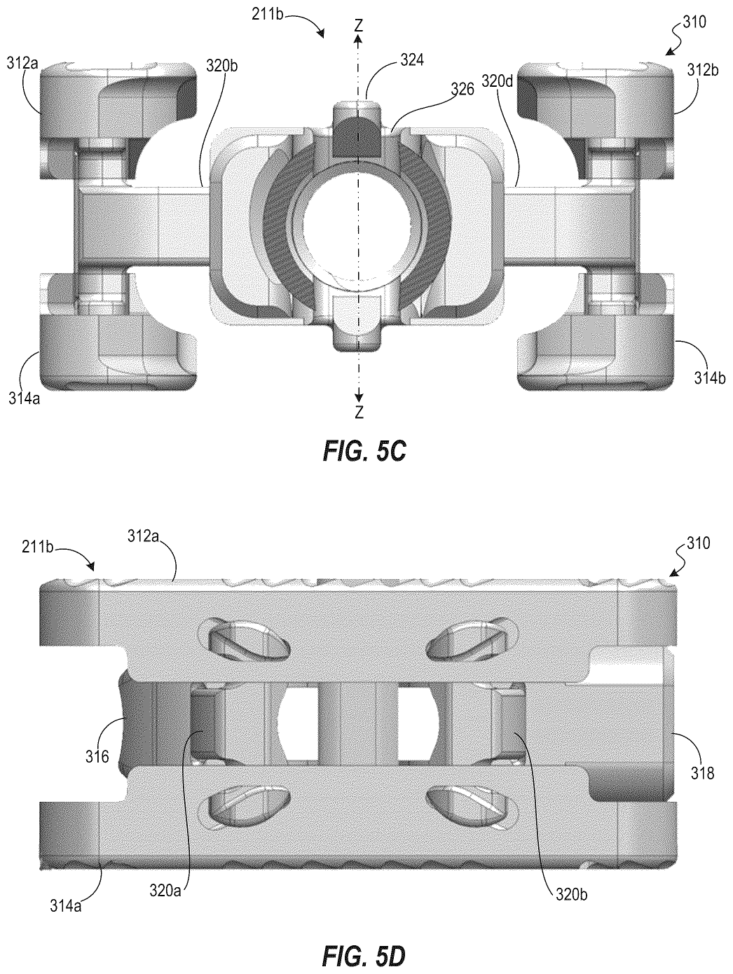

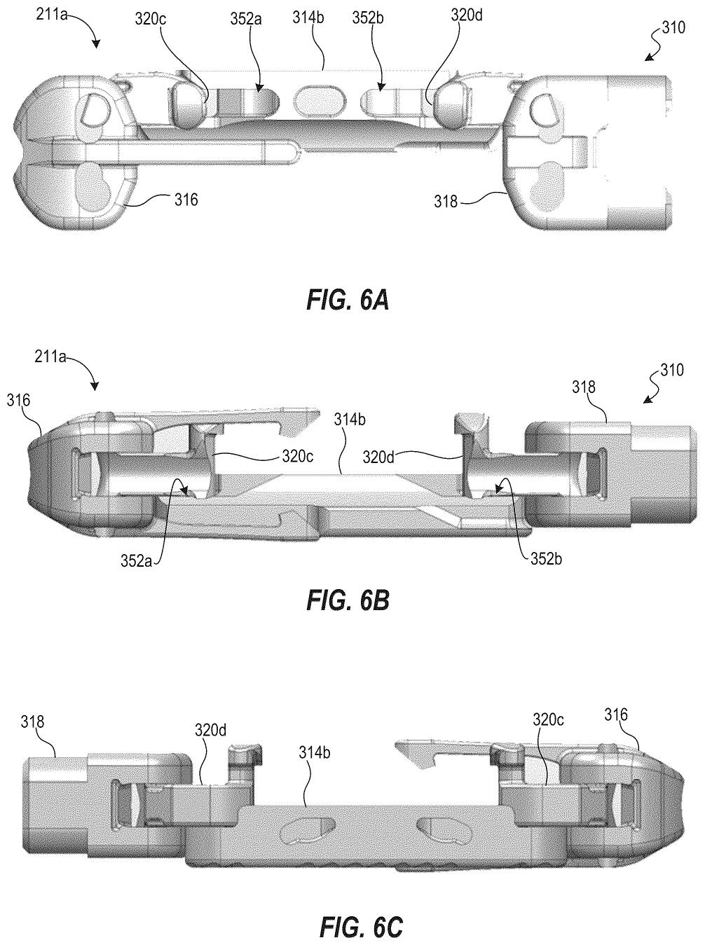

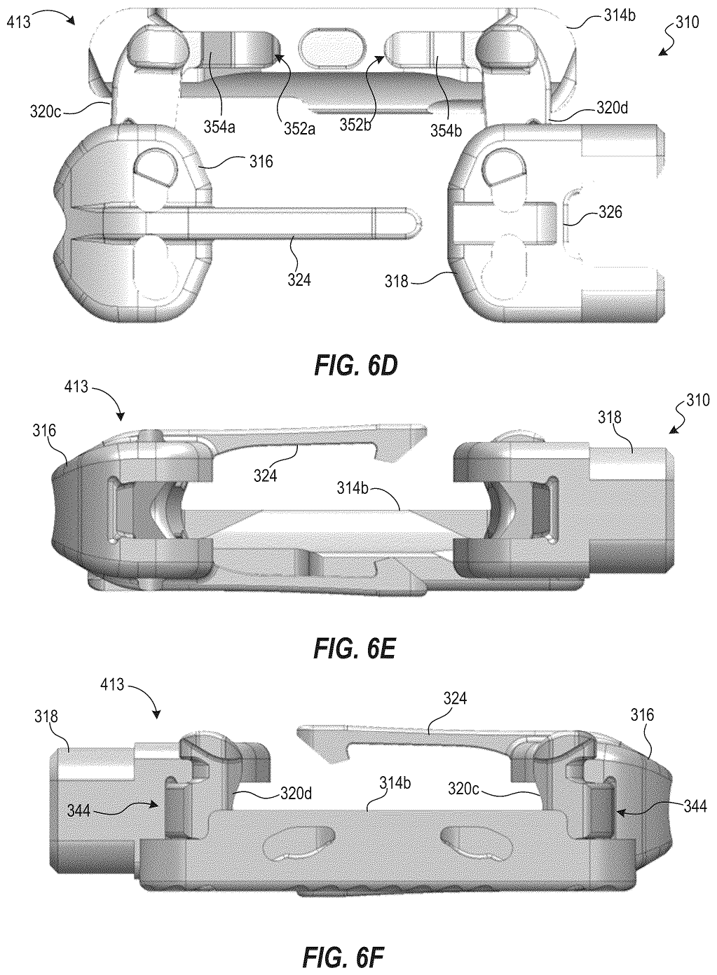

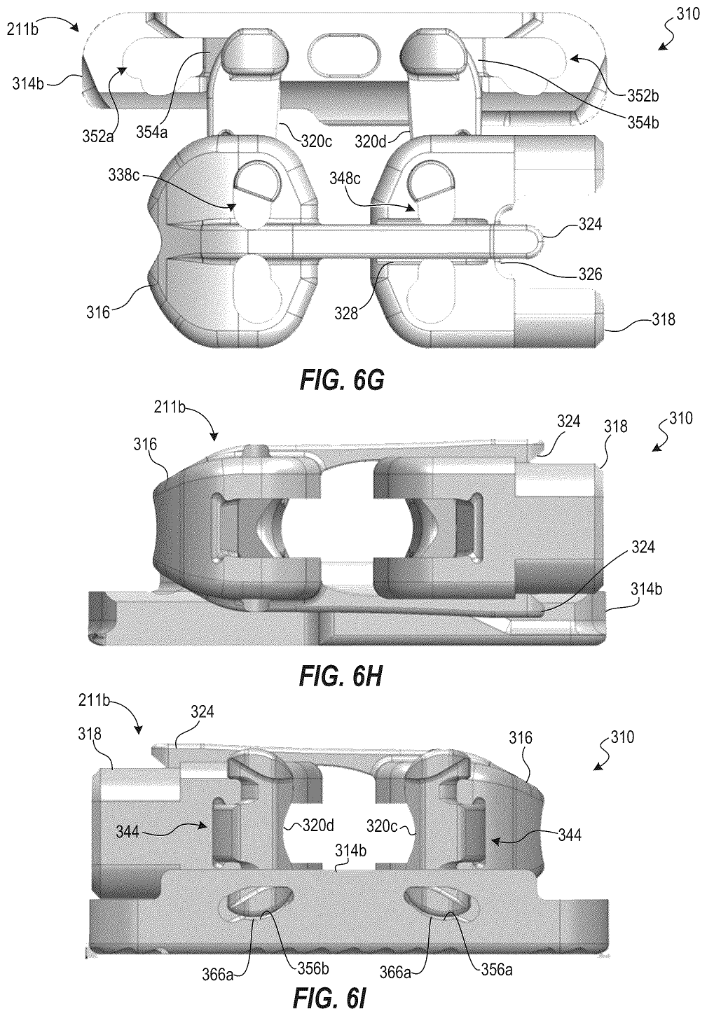

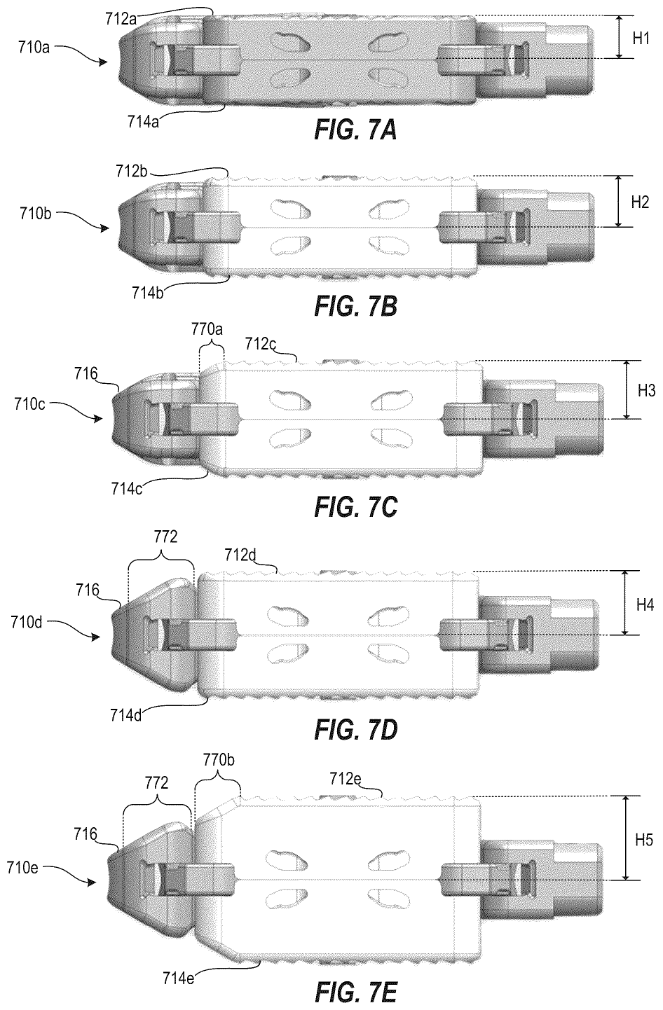

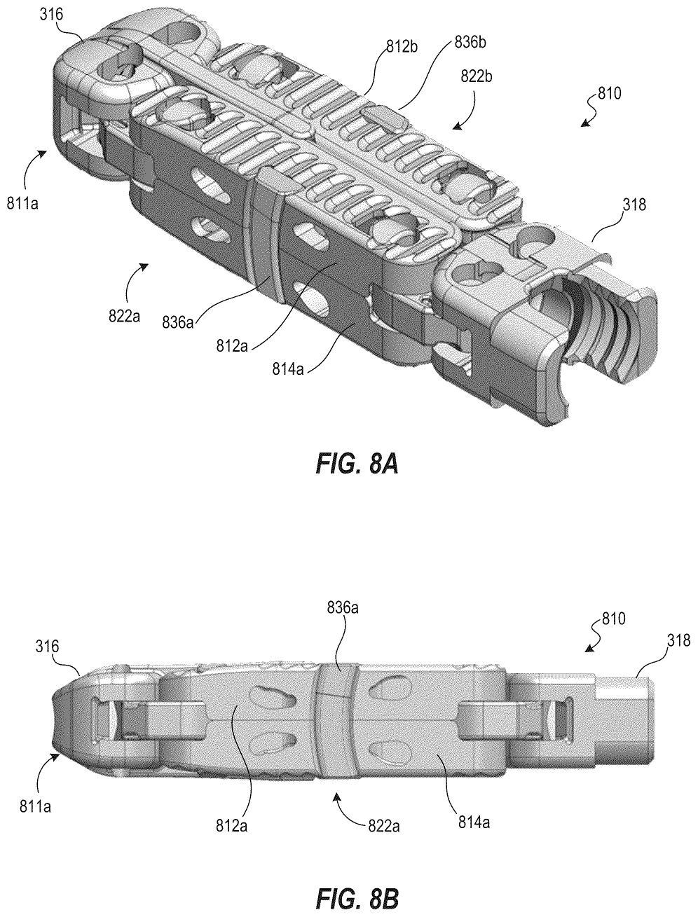

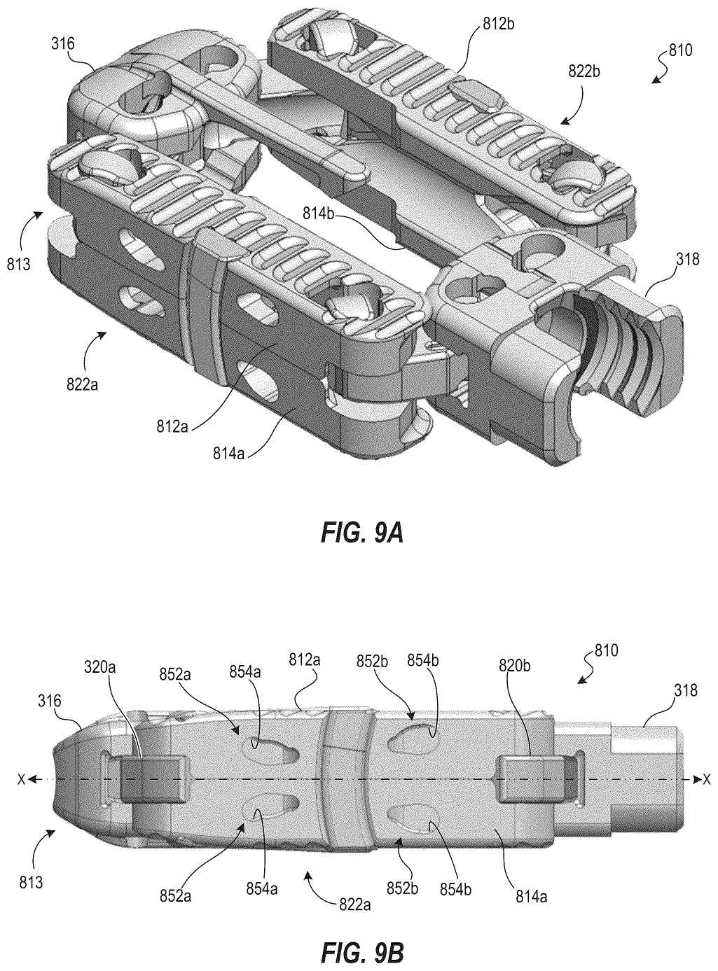

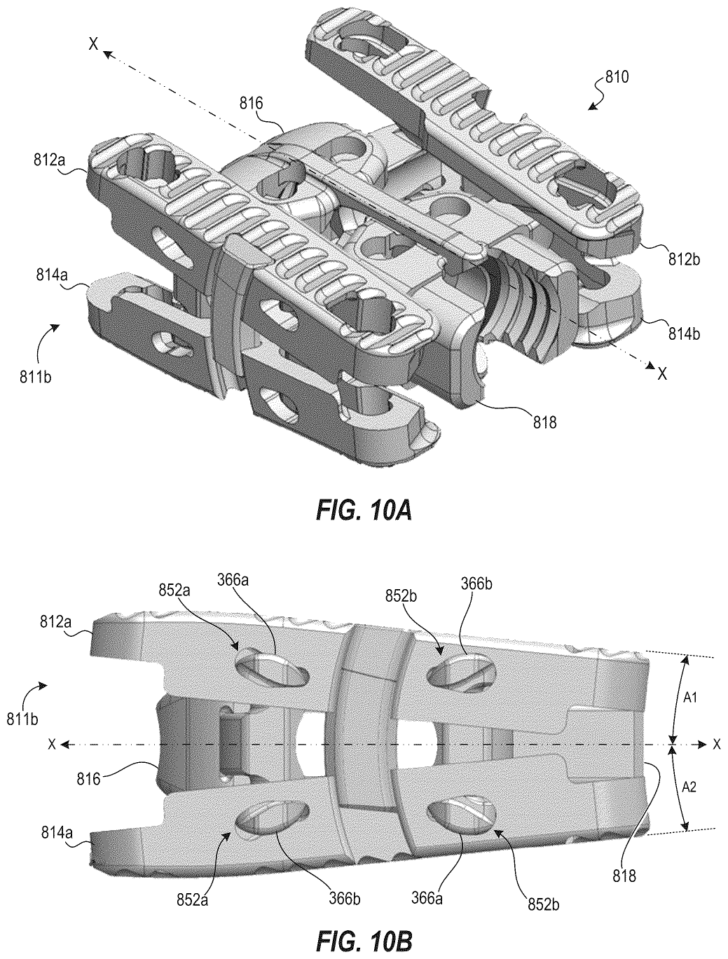

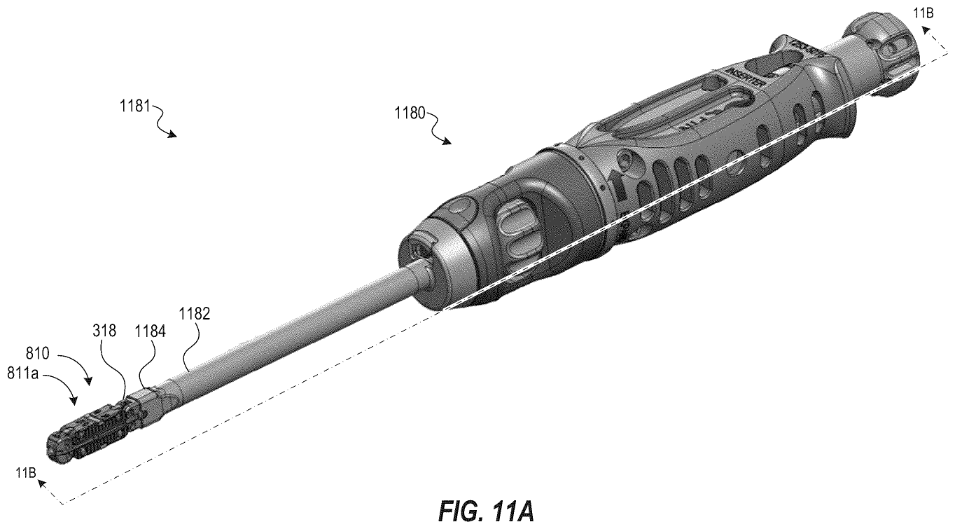

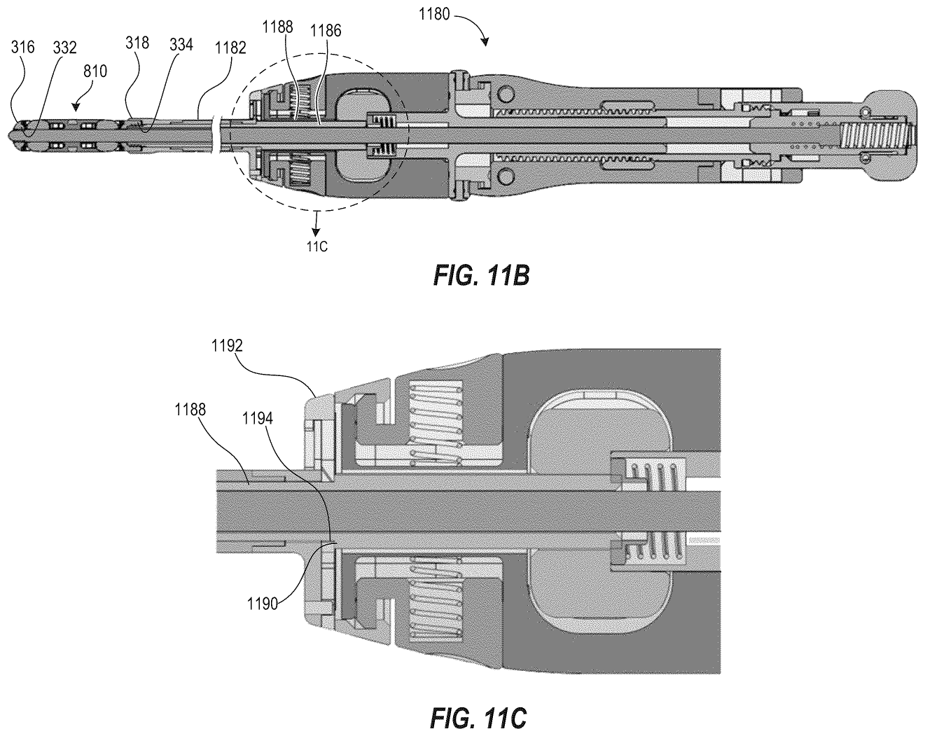

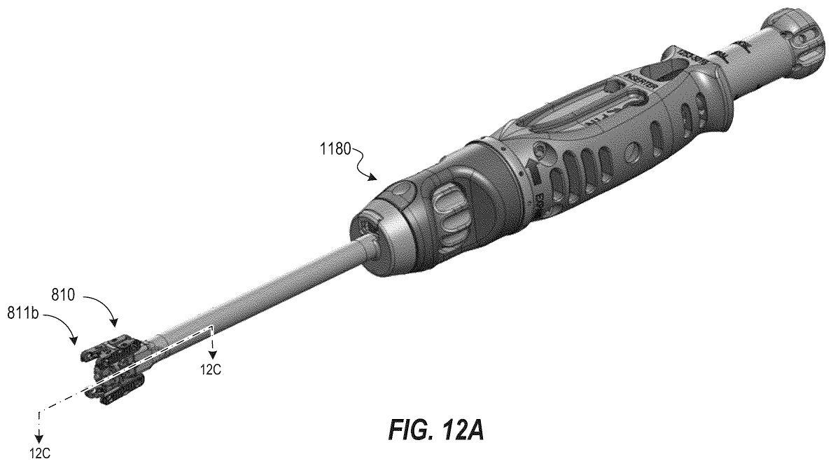

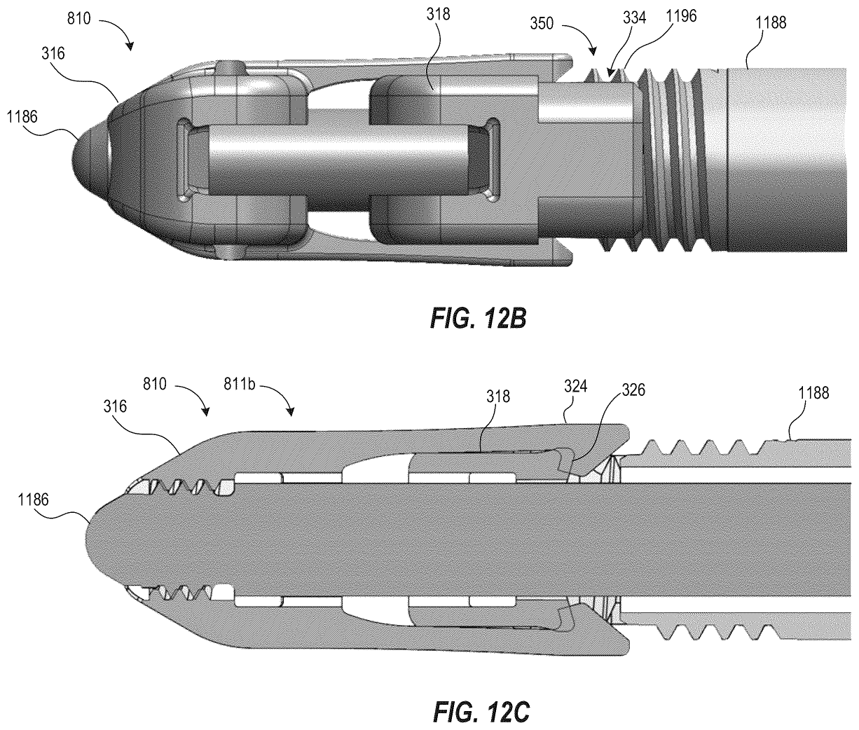

FIGS. 3K and 3L are a perspective view and a side view, respectively, of a support of the intervertebral device of FIG. 3A.

FIGS. 3M-30 are two perspective views and an end view, respectively, of a link of the intervertebral device of FIG. 3A.

FIG. 4A is a perspective view of the intervertebral device of FIG. 3A in a laterally expanded state in accordance with embodiments of the present technology; FIG. 4B is a top view of the intervertebral device of FIG. 4A; FIG. 4C is an end view of the intervertebral device of FIG. 4A; and FIG. 4D is a side view of the intervertebral device of FIG. 4A.

FIG. 5A is a perspective view of the intervertebral device of FIG. 3A in a laterally and vertically expanded state in accordance with embodiments of the present technology; FIG. 5B is a top view of the intervertebral device of FIG. 5A; FIG. 5C is an end view of the intervertebral device of FIG. 5A; and FIG. 5D is a side view of the intervertebral device of FIG. 5A.

FIGS. 6A-6C are a top view and two side views, respectively, of select aspects of the intervertebral device of FIG. 3A, with other aspects of the intervertebral device omitted for illustrative clarity.

FIGS. 6D-6F are a top view and two side views, respectively, of select aspects of the intervertebral device of FIG. 4A, with other aspects of the intervertebral device omitted for illustrative clarity.

FIGS. 6G-6I are a top view and two side views, respectively, of select aspects of the intervertebral device of FIG. 5A.

FIGS. 7A-7E are side views of respective intervertebral devices, in accordance with embodiments of the present technology.

FIGS. 8A and 8B are perspective and side views, respectively, of another intervertebral device in a delivery configuration, in accordance with embodiments of the present technology.

FIGS. 9A and 9B are perspective and side views, respectively, of the intervertebral device of FIGS. 8A and 8B in an laterally expanded state, in accordance with embodiments of the present technology.

FIGS. 10A and 10B are perspective and side views, respectively, of the intervertebral device of FIGS. 8A and 8B in a second state 811b, in accordance with embodiments of the present technology.

FIGS. 11A-11C are perspective and cross-sectional views, respectively, of the intervertebral device of FIGS. 8A and 8B and a delivery tool when the intervertebral device is in the first state.

FIGS. 12A-12C are perspective and cross-sectional views, respectively, of the intervertebral device of FIGS. 8A and 8B and the delivery tool of FIGS. 11A-11C when the intervertebral device of FIGS. 8A and 8B is in the second state.

FIG. 13A-13D are perspective and cross-sectional views, respectively, of the intervertebral device of FIGS. 8A and 8B and the delivery tool of FIGS. 11A-11C after the delivery tool has been actuated to transition the intervertebral toward the intermediate state.

### DETAILED DESCRIPTION

The present technology is generally directed to expandable intervertebral devices, and associated devices, systems, and methods. In some embodiments, an intervertebral device is configured to transition between multiple different states, for example, between a first state (e.g., an unexpanded state, a low-profile state, a delivery state, etc.), an intermediate state (e.g., a partially expanded state, a laterally expanded state, a horizontally expanded state, etc.), and/or a second state (e.g., an expanded state, a lordotically-expanded state, a vertically and horizontally expanded state, an implanted state, etc.). The expansion of the intervertebral device can include, for example, horizontal/lateral expansion, vertical/lordotic expansion, unilateral expansion, bilateral expansion, combinations thereof. In some embodiments, the intervertebral device is configured to expand in stages, for example, to undergo sequential expansion, such as vertical expansion after expanding horizontally/laterally. In some embodiments, the intervertebral device is configured to expand/contract in stages, such as one or more stages of concurrent expansion/contraction (e.g., vertical expansion/contraction and horizontal/lateral expansion/contraction), stages of sequential expansion, or the like. The number, order, and movement (e.g., expansion/contraction) of the stages can be selected based on the procedure to be performed.

The intervertebral device can include locking features that secure or “lock” the intervertebral device in an expanded state, e.g., to inhibit or prevent the intervertebral device from transitioning away from and/or out of the expanded state. The locking features can be integral to (e.g., part of) the intervertebral device. Intervertebral devices that include locking features are expected to be easier to use at least because such intervertebral device can be secured in the expanded state without using externally supplied locking components, such as locking screws. This can reduce the number of tools and/or components inserted into the subject during an implantation procedure, which is expected to increase the speed at which the implantation procedure can be performed, and/or at least partially reduce the risk of damage to the subject's tissue. Additionally, or alternatively, because the intervertebral devices of the present technology can be used without a locking screw, bone graft material (and/or other spinal treatment material) can be delivered through the intervertebral devices without clearing a path for the locking screw; this is expected to further increase the speed of the implantation procedure and/or reduce the number of tools and/or components inserted into the subject during the implantation procedure.

The intervertebral device can be transitioned between states in situ, e.g., after placement in an intervertebral space, or at other anatomical location. The intervertebral device can be configured for vertebral procedures to treat, for example, fractures (e.g., compression fractures), restoring vertebral dimensions (e.g., vertebral height), and/or the like. In at least some embodiments, the intervertebral device can be expanded to at least partially occupy the intervertebral space, e.g., to extend between adjacent vertebral bodies. In the expanded state, the intervertebral device can contact one or more surfaces defining the intervertebral space, such as an inferior surface of a superior vertebral body and/or a superior surface of an inferior vertebral body, and/or one or more portions thereof. In some embodiments, the intervertebral device can assume a curved or angled configuration in the expanded state, e.g., to at least partially correct a lordotic angle of a subject's spine and/or otherwise adjust the relative position and/or alignment of one or more vertebral bodies of the subject's spine.

A delivery tool, drive instrument, actuation mechanism, etc., can be used to deploy the intervertebral device in the intervertebral space and/or lock the intervertebral device in the expanded state. In at least some embodiments, the delivery tool can be configured to transition the intervertebral device from the unexpanded state to the expanded state, and/or unlock the intervertebral device's locking feature to thereby allow the intervertebral device to be transitioned from the expanded state toward the unexpanded state.

### A. Overview of Subject Anatomy

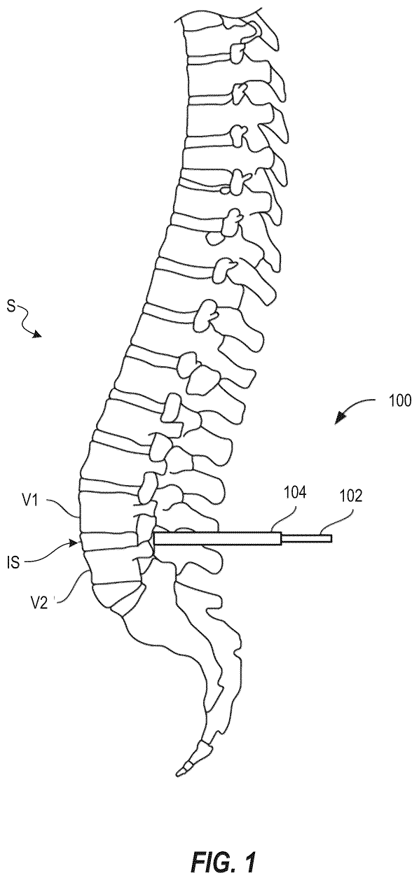

FIG. 1 is a side view of a spinal surgical system 100 (“system 100”) positioned along a human subject's spine S in accordance with an embodiment of the disclosure. The system 100 can include one or more instruments 102 and a retractor or cannula 104 (“retractor 104”). One or more of the instruments 102 can be delivered through the cannula 104 to perform a surgical procedure. For example, at least some of the instruments 102 can be configured to carry an intervertebral device (which can also be referred to as an interbody device, an interbody spacer, an interbody cage, and/or the like), such as any of the intervertebral devices described and/or incorporated by reference herein. The retractor 104 can be configured to provide access via the different paths, e.g., ALIF path, OLIF path, LLIF path, XLIF path, TLIF path, and/or PLIF path.

With continued reference to FIG. 1, the instrument 102 can be used to prepare an implantation site, for example, by moving organs or tissue (e.g., moving nerve tissue), removing tissue (e.g., removing marrow or intervertebral tissue/discs, removing tissue contributing to stenosis, penetrating tissue, etc.), preparing vertebral bodies (e.g., roughening or shaping vertebral endplates or intervertebral surfaces), or the like. In some embodiments, the instrument 102 includes a delivery instrument. The delivery instrument can be inserted into an intervertebral space IS between two vertebrae V (individually identified as a first or superior vertebra V1 and a second or inferior vertebra V2). The intervertebral space (“IS”) can be formed or enlarged using one or more instruments, including bone drills, bone dissectors, scalpels, knifes, cutting instruments, etc. An intervertebral device can be delivered through the retractor 120 and into the intervertebral space. In some procedures, the instrument 110 can be configured to deliver the intervertebral device through the retractor 104.

### B. Select Embodiments of Interbody Devices, and Associated Systems and Methods

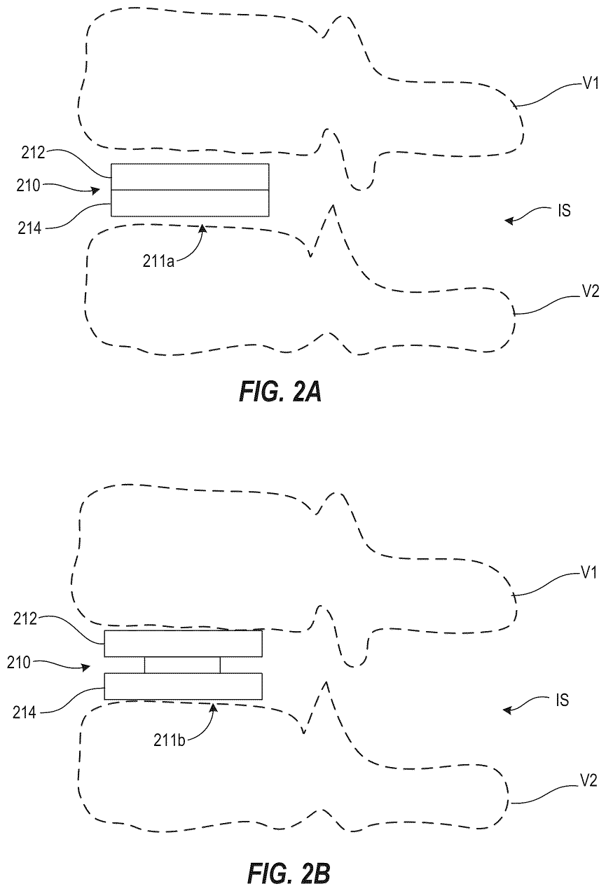

FIGS. 2A and 2B are side views of the vertebral bodies V1, V2 of FIG. 1 and an intervertebral device 210 (“device 210”) positioned within the intervertebral space IS, in accordance with embodiments of the present technology. More specifically, in FIG. 2A the device 210 is in a first state 211a (which can also be referred to as a delivery state, a collapsed state, a compact state, a low-profile state, an unexpanded state, and/or the like) and, in FIG. 2B, the device 210 is in a second state 211b (which can also be referred to as a deployed state, an expanded state, and/or the like). In some embodiments, the device 210 is configured to transition to one or more intermediate or partially expanded states, e.g., between the first state 211a and the second state 211b.

The device 210 can include one or more upper supports 212 and one or more lower supports 214. In the first state 211a, individual ones of the upper and lower supports 212, 214 can be spaced apart from one or both of the vertebral bodies V1, V2. In the second state 211b, individual ones of the upper and lower supports 212, 214 can contact at least one of the vertebra bodies V1, V2. In the illustrated embodiment, for example, one or more of the upper supports 212 contact the first vertebral body V1 and one or more of the lower supports 214 contact the second vertebral body V2 when the device 210 is in the second state 211b. In the second state 211b, the device 210 can be configured to treat the subject's spine, for example, by at least partially correcting an alignment of one or both of the vertebral bodies V1, V2, and/or by stabilizing the positions of the vertebral bodies V1, V2 relative to one another.

FIGS. 3A-3F are views of an intervertebral device 310 (“device 310”) configured in accordance with embodiments of the present technology. More specifically, FIG. 3A is a perspective view of the device 310 in the first state 211a, FIG. 3B is an end view of the device 310, FIG. 3C is another end view of the device 310, FIG. 3D is a side view of the device 310, FIG. 3E is a top view of the device 310, and FIG. 3F is an exploded view of the device 310. At least some aspects of the device 310 can be generally similar or identical in structure and/or function to the device 210 of FIGS. 2A and 2B. Accordingly, like names and/or reference numbers (e.g., the upper supports 212 versus upper supports 312) are used to indicate generally similar or identical aspects.

Referring to FIGS. 3A-3F together, the device 310 can include one or more upper supports 312 (individually identified as a first upper support 312a and a second upper support 312b in FIGS. 3A-3F), one or more lower supports 314 (individually identified as a first lower support 314a and a second lower support 314b in FIGS. 3A-3F), a first or front end body or assembly 316 (“first end body 316”), a second or rear end body or assembly 318 (“second end body 318”), and one or more links or transition members 320 (individually identified as a first link 320a, a second link 320b, a third link 320c, and a fourth link 320d). In the first state 211a, the upper and lower supports 312, 314 can be positioned between the first and second end bodies 316, 318. Individual ones of the links 320 can couple (e.g., moveably couple, pivotally couple, etc.) at least one of the first or second end bodies 316, 318 to at least one of the upper or lower supports 312, 314. In the illustrated embodiment, for example, the first link 320a couples the first end body 316 to the first upper and lower supports 312a, 314a, the second link 320b couples the second end body 318 to the first upper and lower supports 312a, 314a, the third link 320c couples the first end body 316 to the second upper and lower supports 312b, 314b, and the fourth link 320d couples the second end body 318 to the second upper and lower supports 312b, 314b. The first link 320a, the second link 320b, the first upper support 312a, and the first lower support 314a can together comprise a first expansion assembly 322a configured to be movable relative to the first and second end bodies 316, 318. Likewise, the third link 320c, the fourth link 320d, the second upper support 312b, and the second lower support 314d can together comprise a second expansion assembly 322b configured to be movable relative to the first and second end bodies 316, 318. The movement of the expansion assemblies 322a, 322b can cause the device 310 to expand, as described in further detail herein (e.g., with reference to FIGS. 4A-6I).

The first end body 316 can include one or more first locking features or regions 324 and a first attachment port or opening 332. The second end body 318 can include one or more second locking features or regions 326 and a second attachment port or opening 334. Individual ones of the first and second locking features 324, 326 can correspond to and be configured to releasably couple to one another. In the illustrated embodiment, for example, individual ones of the second locking features 326 include a notch or recess configured to releasably receive a corresponding tab or protrusion of one of the first locking features 324. In some embodiments, the second end body 318 can include a sloped surface or region 328 positioned between the first and second locking features 324, 326 at least when the device 310 is in the first state 211a. The sloped surface 328 can be configured to facilitate the coupling of the first and second locking features 324, 326. Additionally, or alternatively, individual ones of the first locking features 324 can be positioned at least partially between one or more of the supports 312, 314, such as at least when the device 310 is in the first state 211a. In the illustrated embodiment, for example, the first and second upper supports 312a, 312b define a recess or first locking feature gap 330 configured to receive at least one of the first locking features 324 when the device 310 is in the first state 211a. In these and other embodiments, the first and second attachment openings 332, 334 can be aligned to define a lumen or central passage through the device 310, such as shown in FIGS. 3B and 3C.

In some embodiments the device 310 can include one or more alignment pegs or posts 336 (individually identified as a first post 336a and a second post 336b in FIGS. 3A-3F). Individual ones of the posts 336 can be positioned at least partially between one or more of the supports 312, 314 and configured to align (e.g., maintain alignment of) at least those supports 312, 314 relative to each other as the device 310 expands from the first state 211a. In the illustrated embodiment, for example, the first post 336a is positioned at least partially between and configured to align the first upper and lower supports 312a, 314a, and the second post 336b is positioned at least partially between and configured to align the second upper and lower supports 312b, 314b.

FIGS. 3G and 3H are perspective and side views, respectively, of the first end body 316. The first end body 316 can include one or more link ports 338 (individually identified as a first link port 338a, a second link port 338b, a third link port 338c, and a fourth link port 338d in FIG. 3G). Each of the link ports 338 can be configured to pivotally couple to one of the links 320, as described previously with reference to FIGS. 3A-3F.

Additionally, the first end body 316 can include an upper first locking feature 324a positioned on a first (e.g., upper) side of the first end body 316, and a lower first locking feature 324b positioned on a second (e.g., lower) side of the first end body 316. Each of the first locking features 324 can include an arm or extension portion 340a-b and a coupling, barb, hook, or tab portion 342a-b. Each of the tab portions 342a-b can be configured to engage a corresponding one of the second locking features 326, as described in further detail below with reference to FIGS. 5A-6I. In the illustrated embodiment, at least a portion of the second end body 318 (FIGS. 3A-3F) can be positioned between the pair of upper and lower first locking features 324a, 324b, and the pair of the upper and lower first locking features 324a, 324b can be configured to pinch and/or clamp onto opposite sides of the second end body 318.

The first end body 316 can further include one or more link lock features 344. Although only one link lock feature 344 is shown on a third (e.g., left) side of the first end body 316, the first end body 316 can include another link lock feature 344 on a fourth (e.g., right) side of the first end body opposite the first side. Each of the link lock features 344 can include one or more registration tabs or protrusions 346 (individually identified as a first registration tab 346a and a second registration tab 346b in FIGS. 3G and 3H). In the illustrated embodiment, the first and second registration tabs 346a, 346b are positioned on opposite sides of the link lock feature 344. As described in further detail herein (e.g., with reference to FIGS. 6D-6I), the link lock features 344 can be configured to inhibit or prevent the device 310 from returning to the first state 211a, e.g., after the device 310 has been expanded from the first state 211a.

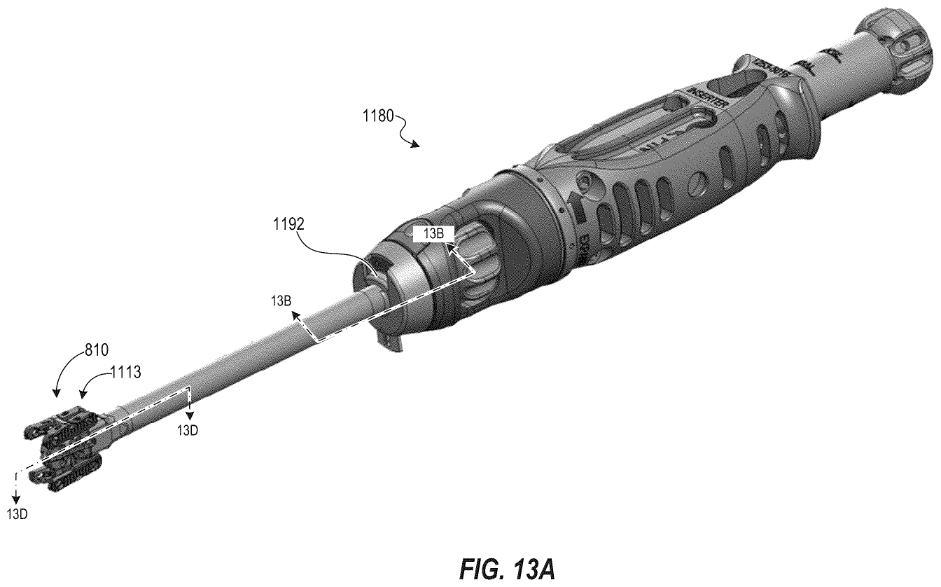

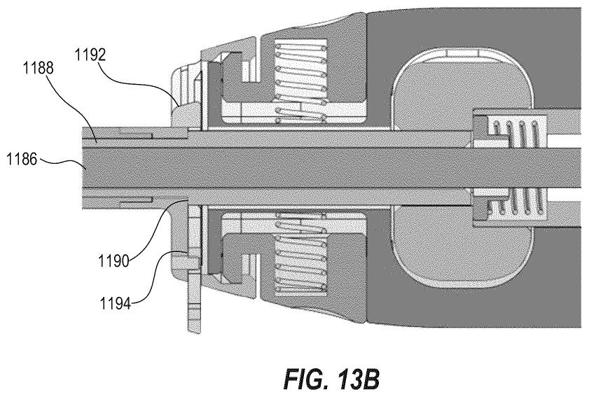

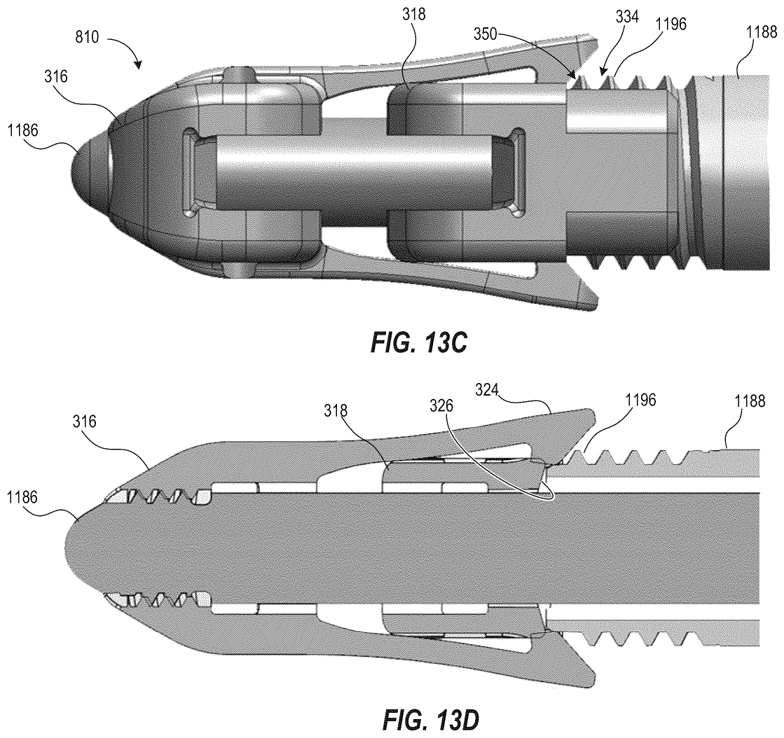

FIGS. 31 and 3J are perspective and side views, respectively, of the second end body 318. The second end body 318 can include one or more of the link lock features 344 described previously herein (e.g., with reference to FIGS. 3G and 3H). Additionally, the second end body 318 can include one or more link ports 348 (individually identified as a first link port 348a, a second link port 348b, and a third link port 348c in FIG. 3G). Although not visible in FIG. 31, the second end body 318 can further include a fourth link port opposite the third link port 348c. Each of the link ports 348 can be at least generally similar or identical in structure and/or function to one or more of the link ports 338 described previously herein (e.g., with reference to FIGS. 3G and 3H) configured to pivotally couple to one of the links 320, as described previously with reference to FIGS. 3A-3F. Each of the second coupling features 326 can include a notch or recessed region configured to receive a corresponding one of the first coupling features 324. Although only one (e.g., upper) second locking feature 326 and one corresponding (e.g., upper) sloped surface 328 are visible in the embodiment illustrated in FIG. 31, the second end body 318 can include another second (e.g., lower) coupling feature and a corresponding second (e.g., lower) sloped surface, each positioned on an opposite (e.g., lower) side of the second end body 318 relative to the illustrated second locking feature 326 and sloped surface 328. As best shown in FIG. 31, the attachment port 334 can include a gap 350, which can divide the attachment port 334 into portions or halves (e.g., left and right portions/halves) positioned on either side of the gap 350.

FIGS. 3K and 3L are perspective and side views, respectively, of the second lower support 314b. A person of ordinary skill in the art will appreciate that one or more of the other supports 312, 314 can be at least generally similar or identical in structure and/or function to the second lower support 314b. For example, the first lower support 314a and/or the second upper support 312b can be mirror images of the second lower support 314b (e.g., about a longitudinal axis of the device 310), and/or the first upper support 312a can be identical to the second lower support 314b.

The second lower support 314b can include one or more expansion slots 352 (individually identified as a first expansion slot 352a and a second expansion slot 352b in FIGS. 3K and 3L). The second expansion slot 352b can be a mirror image of the first expansion slot 352a. Each of the expansion slots 352a-b can be configured to receive and be pivotally coupled to one or more of the links 320. For example, the first expansion slot 352a is configured to receive and be pivotally coupled to the third link 320c (e.g., FIG. 3F), and the second expansion slot 352b is configured to receive and be pivotally coupled to the fourth link 320d (e.g., FIG. 3F). Each of the expansion slots 352 can include a ramped surface 354a-b. As described in further detail below with reference to FIGS. 6A-6I, movement of the links 320 through the corresponding expansion slots 352 and/or along the associated ramped surfaces 354a-b can cause the device 310 to expand from the first state 211a. Each of the expansion slots 352 can further include a recess or detent 356a-b configured to releasably couple to one of the links 320 when the device 310 is in a second (e.g., expanded) state.

The second lower support 314b can further include an alignment port 358, which can be positioned between the first and second expansion slots 352a-b and can be configured to receive a corresponding one of the alignment posts 336. In the illustrated embodiment, for example, the alignment port 358 of the second lower support 314b is configured to receive the second alignment post 336b (e.g., FIG. 3F). Additionally, the second lower support 314b can include a recessed portion 360 that at least partially defines (e.g., defines a first half of) the first locking feature gap 330.

FIGS. 3M-30 are a first perspective view, a second perspective view, and an end view, respectively, of one of the links 320. Each of the links 320 can include one or more end body coupling features 362 (individually identified as an upper end body coupling feature 362a and a lower end body coupling features 362b in FIGS. 3M and 3N) and one or more support coupling features 364 (individually identified as an upper support coupling feature 364a and a lower support coupling features 364b in FIGS. 3M-30). The end body coupling features 362 and/or the support coupling features 364 can be integrated into the links 320, e.g., to form a unitary, single-piece assembly, which can reduce the number of individual components in the device 310. Each of the end body coupling features 362 can be configured to be received by one of the link ports 338, 348 of the first or second end bodies 316, 318 (FIGS. 3G-3J). Each of the support coupling features 364 can be configured to be received by one of the expansion slots 352 of the supports 312, 314 (FIGS. 3K and 3L). Optionally, one or more of the support coupling features 364 can include a curved or rounded head portion 366a, 366b configured to correspond to and be positionable at least partially within one of the detents 356a, 356b (FIG. 3L), as described in further detail herein (e.g., with reference to FIGS. 4B and 6I). In some embodiments, the link 320 can include one or more registration slots 368 (individually identified as a first registration slot 368a and a second registration clot 368b in FIG. 3N). Each of the registration slots 368 can be configured to receive a corresponding one of the registration tabs 346 of one of the link lock features 344 (FIGS. 3G-3J), as described in detail herein (e.g., with reference to FIG. 6D).

FIGS. 4A-4D are views of the device 310 in an intermediate state 422c, e.g., between the first state 211a and the second state 211b. More specifically, FIG. 4A is a perspective view of the device 310, FIG. 4B is a top view of the device 310, FIG. 4C is an end view of the device 310, and FIG. 4D is a side view of the device 310. In the intermediate state 413, relative positions of one or more features of the device 310 can be different than in the first state 211a. In the illustrated embodiment, for example the first and second end bodies 316, 318 have been moved toward each other, e.g., along a longitudinal axis X of the device 310, such that the device 310 is shorter and/or longitudinally compressed in the intermediate state 422c relative to the first state 211a. This movement of the first and second end bodies 316, 318 can position the first and second locking features 324, 326 closer to one another, e.g., without causing the first and second locking features to couple to one another. With continued reference to the illustrated embodiment, individual ones of the upper and lower supports 312, 314 have been moved away from each other, e.g., along a lateral or width-wise axis Y of the device 310, such that the device 310 is widened or laterally expanded relative to the first state 211a. For example, the first upper and lower supports 312a, 314a have been moved in a first direction away from the longitudinal axis X, and the second upper and lower supports 312b, 314b have been moved in a second direction away from the longitudinal axis X opposite the first direction. Individual ones of the links 320 can pivot relative to one or more of the end bodies 316, 318 and/or one or more of the supports 312, 314 in response to the relative movement of the end bodies 316, 318 and the supports 312, 314. As described in further detail herein (e.g., with reference to FIGS. 6A-6I), the movement of the links 320 can drive the horizontal expansion of the device 310.

FIGS. 5A-5D are views of the device 310 in the second state 211b. More specifically, FIG. 5A is a perspective view of the device 310, FIG. 5B is a top view of the device 310, FIG. 5C is an end view of the device 310, and FIG. 5D is a side view of the device 310. In the second state 211b, the relative positions of one or more features of the device 310 can be different than in the first state 211a and/or the intermediate state 413. In the illustrated embodiment, for example the first and second end bodies 316, 318 have been moved toward each other, e.g., relative to their respective positions in the intermediate state 413, and/or to position the first and second locking features 324, 326 to couple to one another. With continued reference to the illustrated embodiment, individual ones of the upper and lower supports 312, 314 have been moved away from each other, e.g., along a vertical or height-wise axis Z of the device 310, such that the device 310 is taller or vertically expanded relative to the first state 211a and/or the intermediate state 413. For example, the first upper and lower supports 312a, 314a have been moved away from each other in opposite directions along the vertical axis Z, and the second upper and lower supports 312b, 314b have been moved away from each other in opposite direction along the vertical axis Z. Individual ones of the links 320 can move relative to one or more of the supports 312, 314 in response to the movement of the end bodies 316, 318. As described in further detail herein (e.g., with reference to FIGS. 6A-6I), the movement of the links 320 can drive the vertical expansion of the device 310.

FIGS. 6A-6I are views of select aspects of the device 310 at various stages of expansion, with other aspects of the device 310 omitted for the purpose of clarity. More specifically, FIGS. 6A-6C are a top view and two side views of the device 310 in the first state 211a, FIGS. 6D-6F are a top and two side views of the device 310 in the intermediate state 413, and FIGS. 6G-6I are a top and two side views of the device 310 in the second state 211b. Referring to FIGS. 6A-6F together, as the device 310 transitions from the first state 211a to the intermediate state 413, the first and second end bodies 316, 318 can move inwardly toward one other. Because the third and fourth links 320c-d pivotally couple the first and second end bodies 316, 318 to the second lower support 314b (via, e.g., respective expansion slots 352a-b), the inward movement of the first and second end bodies 316, 318 can cause the third and fourth links 320c-d to pivot relative to the first and second end bodies 316, 318 and the second lower support 314b to thereby move the second lower support 314b away from the first and second end bodies 316, 318 and transition the device 310 from the first state 211a toward the intermediate state 413.

Referring to FIGS. 6D-6I together, further movement of the first and second end bodies 316, 318 can cause the device 310 to transition from the intermediate state 413 toward and/or to the second state 211b. In the intermediate state 413, the link lock features 344 of the first and second end bodies 316, 318 can engage the third and fourth links 320c, 320d (best shown in FIG. 6I) to thereby inhibit or prevent further rotation of the third and fourth links 320c, 320d relative to the first and second end bodies 316, 318. For example, the registration tabs 346 (FIGS. 3G-3J) of the link lock features 344 can be positioned at least partially within the registration slots 368 (FIGS. 3M-30) of the links 320. Accordingly, further movement of the first and second end bodies 316, 318 toward one another can cause the third and fourth links 320c, 320d to move along the corresponding expansion slots 352a-b. This movement of the third and fourth links 320c, 320d can drive the third and fourth links 320c, 320d into contact with and along the ramped surface 354a-b of the associated expansion slot 352a-b and thereby cause vertical movement (e.g., expansion) of the second lower support 314b relative to the third and fourth links 320c, 320d. Accordingly, the device 310 can undergo vertical expansion after (e.g., only after) having undergone horizontal expansion, which is expected to reduce or prevent contact between the vertebrae/spinal cord and nerve roots, for example, by maintaining the curvature of at least a portion of the subject's spine (e.g., when the intervertebral device is in the second state 211b) while increasing the distance between individual vertebral bodies. Additionally or alternatively, the device 310, can undergo horizontal collapse after (e.g., only after) the device 310 has undergone vertical/lordotic collapse, which can help maintain the device 310 in the horizontally-expanded state during a procedure to reposition the device 310, e.g., when vertical/lordotic collapse is desired but it is advantageous to maintain the device in the horizontally-expanded state. Because the link lock features 344 can inhibit or prevent further rotation of the third and fourth links 320c, 320d relative to the first and second end bodies 316, 318, the device 310 is expected to be transitionable between the intermediate state 413 and the second state 211b while maintaining horizontal expansion of the device 310. Accordingly, the device 310 can be repositioned within the subject without or substantially without having to return the device 310 to the first state 211a. This, in turn, can increase the speed with which a user can reposition the device 310 during an implantation procedure.

Additionally, moving the first and second end bodies 316, 318 toward one another can cause the first locking features 324 to couple to the corresponding second locking features 326 and thereby secure or lock the device in the second state 211b. For example, referring to FIG. 6G, moving the first and second end bodies 316, 318 toward one another can cause the first locking features 324 to be driven against the corresponding ramped surfaces 328 of the second end body 318. The slope/curvature of the ramped surfaces 328 can bend/deflect the first locking features 324 outwardly (e.g., away from the second end body 318). After passing along the ramped surfaces 328, the first locking features 324 can return to an original (e.g., unbent and/or pre-deflected) state in which the first locking features 324 are positioned to contact the corresponding second locking features 326. In this position, the first and second locking features 324, 326 can inhibit or prevent the device 310 from being transitioned from the second state 211b toward the intermediate state 413 and/or the first state 211a.

In some embodiments, the interaction between one first locking feature 324 and one second locking feature 326 is sufficient to inhibit or prevent the device 310 from being transitioned from the second state 211b toward the intermediate state 413 and/or the first state 211a. In other embodiments, multiple first and second locking features 324, 326 can be used to further inhibit or prevent the device 310 from being transitioned away from the second state 211b. For example, referring to FIG. 6H, the device 310 includes two first locking features 324, e.g., positioned on opposite sides of the first end body 316 and/or configured to pinch or clamp onto corresponding opposite sides of the second end body 318.

Referring to FIG. 6I, in the second state 211b, the movement of the third and fourth links along the ramped surfaces 354a, 354b of the expansion slots 352a, 352b can position the curved heads 366a of the third and fourth links 320c, 320d at least partially within the detents 356a, 356b. The contact between the detents 356a, 356b and the curved heads 366a can further secure or lock the device in the second state 211b, e.g., before, during, and/or after the coupling of the first and second locking features 324, 326. This, in turn, can further inhibit or prevent the device from collapsing or otherwise transitioning away from the second state 211b after expansion/implantation.

FIGS. 7A-7E are side views of respective devices 710a-710e, each configured in accordance with embodiments of the present technology. Each of the devices 710a-710e can be at least generally similar or identical in structure and/or function to the device 310. Referring to FIGS. 7A-7E together, the dimensions of one or more aspects of the devices described herein can be customized based at least partially on a subject's anatomy, such as a size of the intervertebral space IS (FIGS. 1-2B). For example, each of the devices 710a-e include respective upper and lower supports 712a-e, 714a-e having respective heights H1-H5, each subsequent height being greater than the preceding height (e.g., H2>H1, H3>H2, etc.). Additionally, or alternatively, individual ones of the devices 710a-e can include one or more tapered or sloped leading edge portions 770, 772. For example, referring to FIG. 7C, the upper and lower supports 712c, 714c of the device 710c include a tapered leading-edge portion 770a positioned proximate to the first end body 716. The slope/curvature of the tapered leading-edge portion 770a can be configured to create a generally smooth transition between the first end body 716 and the upper and lower supports 712c, 714c of the device 710c, e.g., to reduce the resistance to positioning the device 710c within the intervertebral space IS. As another example, referring to FIG. 7D, the first end body 716 includes a tapered leading-edge portion 772 configured to create a generally smooth transition between the first end body 716 and the upper and lower supports 712d, 714d of the device 710d. As a further example, referring to FIG. 7E, the first end body 716 includes the tapered leading-edge portion 772 and the upper and lower supports 712e, 714e of the device 710e include a tapered leading-edge portion 770b. 710a (FIG. 7C). Both tapered leading-edge portions 770b, 772 can be configured to create a generally smooth transition between the first end body 716 and the upper and lower supports 712e, 714e. Because the height H5 of the upper and lower supports 712e, 714e of the device 710e can be greater than the height H3 of the upper and lower supports 712c, 714c of the device 710c (FIG. 7C), the tapered leading-edge portion 770b of the device 710e can be different (e.g., longer) than the tapered leading-edge portion 770a of the device.

FIGS. 8A-10B are views of another intervertebral device 810 (“device 810”) configured in accordance with embodiments of the present technology. Specifically, FIGS. 8A and 8B are perspective and side views of the device 810 in a first state 811a, FIGS. 9A and 9B are perspective and side views of the device 810 in an intermediate state 813, and FIGS. 10A and 10B are perspective and side views of the device 810 in a second state 811b. At least some aspects of the device 810 can be generally similar or identical in structure and/or function to one or more of the devices 210, 310, 710a-e described previously herein (e.g., FIGS. 2A-7E). Accordingly, like names and/or reference numbers (e.g., first end body 816 versus the first end body 316) are used to indicate generally similar or identical aspects. Additionally, referring to FIGS. 8A and 8B, the alignment pegs 836a-b of the device 810 can include curved and configured to aid the curved expansion of the device 810, as described in further detail herein (e.g., with reference to FIGS. 10A and 10B).

Referring to FIGS. 8A-9B together, the first and second end bodies 316, 318 of the device 810 can be moved inwardly and/or toward one another to transition the device 810 from the first state 811a (FIGS. 8A and 8B) toward and/or to the intermediate state 813 (FIGS. 9A and 9B), as described previously herein (e.g., with reference to FIGS. 6A-6F). This movement of the first and second end bodies 316, 318 can cause a corresponding movement of the expansion assemblies 822a, 822b (e.g., the upper and/or lower supports 812, 814), as described previously herein (e.g., with reference to FIGS. 6A-6F).

Referring to FIGS. 9A-10B together, further movement of the first and second end bodies 316, 318 toward one another can cause the device 810 to transition from the intermediate state 813 (FIGS. 9A and 9B) toward and/or to the second state 811b state (FIGS. 10A and 10B), as described previously herein (e.g., with reference to FIGS. 6D-6I). This movement of the first and second end bodies 316, 318 can cause a corresponding movement of the expansion assemblies 822a, 822b, for example, driving individual ones of the upper and/or lower supports 812, 814 away from one another, as described previously herein (e.g., with reference to FIGS. 6D-6I).

Referring to FIGS. 10A and 10B, in the second state 811b, one or more of the supports 812, 814 can be angled, e.g., relative to the longitudinal axis X of the device 810. In the illustrated embodiment, for example, the first upper support 812a is at a first angle A1 relative to the longitudinal axis X and the first lower support 814a is at a second angle A2 relative to the longitudinal axis X. The second angle A2 can be greater than, less than, or equal to the first angle A1. The first angle A1 and/or the second angle can be between about 1 degree and about 30 degrees, such as at least 1 degree, 2 degrees, 3 degrees, 4 degrees, 5 degrees, 6 degrees, 7 degrees, 8 degrees, 9 degrees, 10 degrees, 15 degrees, 20 degrees, 25 degrees, any angle therebetween, or another suitable angle. In at least some embodiments, the first angle A1 and/or the second angle A2 can be selected based at least partially on a curvature of the subject's spine S (FIG. 1), e.g., such that the device 810 can provide a desired lordotic correction when positioned between the first and second vertebral bodies V1, V2 (FIG. 1). Although in the illustrated embodiment all the supports 812, 814 are configured to be angled when the device 810 is in the second state 811b, in other embodiments a subset of the supports 812, 814 (e.g., the upper supports 812a, 812b or the lower supports 814a, 814b) are configured to be angled when the device 810 is in the second state 811b.

Referring additionally to FIG. 9B, the expansion slots 852a, 852b of the upper and/or lower supports 812, 814 can be configured to drive the angled expansion of the device 810. For example, as best shown in FIG. 9B, the first expansion slot 852a and/or the first ramped surface 854a have a different configuration (e.g., are positioned closed to the longitudinal axis X, have a greater slope or incline, etc.) than the second expansion slot 852b and/or the second ramped surface 854b. Accordingly, when the first link 320a is driven along the first ramped surface 854a, it can lead to a greater amount of movement relative to the movement caused by driving the second link 320b along the second ramped surface 854b, thereby causing the angled vertical/lordotic expansion of the first upper and lower supports 812a, 814a (and/or one or more of the other supports 812, 814). As described previously herein (e.g., with reference to FIGS. 3M-30 and 6I) and shown in FIG. 10B, each of the links 320 can engage the corresponding expansion slots 852 via the rounded link head 366. Accordingly, even though the first and second expansion slots 852a, 852b can have different configurations, the rounded link heads 366 of the links 320 are expected to have improved contact and/or engagement with both the first and second expansion slots 852a, 852b during the angled vertical/lordotic expansion, which can improve the fatigue resistance of the device 310 and/or improve the device's overall durability.

FIGS. 11A-13D are views of the device 810 and an implantable device delivery tool 1180 (“delivery tool 1180”) configured in accordance with embodiments of the present technology. More specifically, FIGS. 11A-11C are perspective and cross-sectional views of the device 810 and the delivery tool 1180 when the device 810 is in the first state 811a, FIGS. 12A-12C are perspective and cross-sectional views of the device 810 and the delivery tool 1180 when the device 810 is in the second state 811b, and FIG. 13A-13D are perspective and cross-sectional views of the device 810 and the delivery tool 1180 after the delivery tool has been actuated to transition the device 810 toward the intermediate state 813. Although FIGS. 11A-13D include the device 810, a person of ordinary skill in the art will understand that the delivery tool 1180 can also be configured for use with the device 310 of FIGS. 3A-6I, or another suitable intervertebral device.

Referring to FIG. 11A, the delivery tool 1180 can include an insertion shaft 1182 having a device-receiving portion 1184 configured to releasably receive the second end body 318 of the device 810. The delivery tool 1180 and the device 810 can together define a spinal system 1181 configured to treat a subject's spine. Referring to FIG. 11B, the delivery tool 1180 can further include a first coupling shaft 1186 configured to be positioned at least partially within and/or coupled (e.g., threadably coupled) to the first attachment opening 332 of the first end body 316, and a second coupling shaft 1188 configured to be positioned at least partially within and/or coupled (e.g., threadably coupled) to the second attachment port 334 of the second end body 318. The first coupling shaft 1186 can be movably positioned within the second coupling shaft 1188, and both the first and second coupling shafts 1186, 1188 can be movably positioned within the insertion shaft 1182. Referring to FIG. 11C, second coupling shaft 1188 can include a notch 1190 configured to contact a stopping tab or feature 1194 of an insertion limiter 1192. When the notch 1190 contacts the stopping tab 1184, the insertion limiter 1192 can be configured to inhibit or prevent movement of the second coupling shaft 1188, e.g., distally and/or toward the first end body 316 (FIG. 11B).

Referring to FIG. 12A, the delivery tool 1180 has been used to transition the device 810 to the second state 811b. For example, referring to FIG. 12B, one or both of the first and second coupling shafts 1186, 1188 can be moved relative to one another to cause a corresponding movement of the first and second end bodies 316, 318 toward one another and thereby cause the device 810 to expand, as described previously herein (e.g., with reference to FIGS. 6A-6l and 8A-10B). As best seen in FIG. 12B, when the second coupling shaft 1188 is coupled to the second attachment port 334 of the second end body 318, at least a portion of the threads 1196 can extend outwardly through the gap 350 defined by the second attachment port 334 (see, e.g., FIG. 31). Referring to FIG. 12C, the movement of the first and second coupling shafts 1186, 1188 can cause the first and second locking features 324, 326 to couple to one another, as described previously herein (e.g., with reference to FIGS. 5A-5D and 6A-6I).

Referring to FIG. 13A, the insertion limiter 1192 of the delivery tool 1180 has been actuated (e.g., depressed) to allow the device 810 to be transitioned to an intermediate state 1113. Referring to FIG. 13B, actuating the insertion limiter 1192 can move the stop tab 1194 out of contact with the notch 1190 of the second coupling shaft 1188, and thereby allow further movement of the second coupling shaft 1188. For example, referring additionally to FIGS. 13C and 13D, in which select aspects of the device 810 are shown and other aspects (e.g., the upper and lower supports) are omitted for illustrative clarity, the second coupling shaft 1188 can be advanced toward (e.g., further toward) the first end body 316 after the insertion limiter 1192 has been actuated. This movement of the second coupling shaft 1188 can cause the second coupling shaft 1188 to contact the first locking feature 324 and thereby uncouple the first and second locking features 324, 326 from one another. In the illustrated embodiment, for example, the second coupling shaft 1188 causes the first locking feature 324 to bend/deflect outwardly (e.g., away from the first and/or second coupling shafts 1186, 1188) and/or out of contact with the second locking feature 326 and thereby allow the first and/or second end bodies 316, 318 to be moved relative to one another, such that the device 810 can be transitioned away from the second state 811b, such as toward and/or to the intermediate state 813 and/or the first state 811a.

### C. Example

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. An intervertebral spacer, comprising:
a first expansion assembly including a first upper support and a first lower support;
a second expansion assembly including a second upper support and a second lower support;
a first end body coupled to the first expansion assembly and the second expansion assembly, the first end body including a first locking feature; and
a second end body coupled to the first expansion assembly and the second expansion assembly, the second end body including a locking region configured to receive the first locking feature when driving apart and expanding the first and second expansion assemblies to move the intervertebral spacer from an unexpanded state to an expanded state, and to cause the first locking feature to couple to the locking region to hold the intervertebral spacer in the expanded state.

2. The intervertebral spacer of example 1 wherein the first locking feature includes a hooked region, wherein the second locking feature includes a notch, and wherein the hooked region is positioned to contact the notch when the intervertebral spacer is in the expanded state.

3. The intervertebral spacer of any one of the examples 1-2 wherein the second end body further includes a ramped surface configured to deflect the first locking feature outwardly from the second end body when the intervertebral spacer is moved from the unexpanded state to the expanded state.

4. The intervertebral spacer of any one of the examples 1-3 wherein the first locking feature is a first upper locking feature configured to couple to a first side of the second end body, the first end body further comprising a first lower locking feature configured to couple to a second side of the second end body opposite the first side when the intervertebral spacer is in the expanded state.

5. The intervertebral spacer of any one of the examples 1˜4 wherein:
in the unexpanded state—
   the first expansion assembly and the second expansion assembly are positioned at least partially between the first end body and the second end body, and
   the first locking feature is spaced apart from the second locking feature, and in the expanded state—
   the first end body and the second end body are positioned at least partially between the first expansion assembly and the second expansion assembly, and
   the first locking feature is coupled to the second locking feature to at least partially prevent the intervertebral spacer from returning to the unexpanded state.

6. The intervertebral spacer of any one of the examples 1-5 wherein in the expanded state, the first upper support and the first lower support are spaced apart from one another, and the second upper support and the second lower support are spaced apart from one another.

7. The intervertebral spacer of any one of the examples 1-6 wherein, in the second state, the first upper support, the first lower support, the second upper support, and/or the second lower support are angled relative to a longitudinal axis of the intervertebral spacer.

8. The intervertebral spacer of any one of the examples 1-7 wherein, in the unexpanded state, the first locking feature is positioned at least partially between the first expansion assembly and the second expansion assembly.

9. The intervertebral spacer of example 8 wherein the first upper support, the first lower support, the second upper support, and/or the second lower support include a recessed portion defining a locking feature gap in which at least a portion of the first locking feature is positioned when the intervertebral spacer is in the unexpanded state.

10. The intervertebral spacer of any one of the examples 1-9, further comprising a plurality of links configured to couple the first and second expansion assemblies to the first and second end bodies, wherein—
individual ones of the links include a rounded link head, and
the first upper support, the first lower support, the second upper support, and/or the second lower support include an expansion slot having a detent configured to receive the rounded link head when the intervertebral spacer is in the expanded state.

11. The intervertebral spacer of example 10 wherein at least one of the plurality of links includes a registration slot configured to mate with a corresponding link lock feature of the first or second end body to at least partially prevent rotational movement of the at least one link relative to the first or second end body.

12. The intervertebral spacer of any one of the examples 1-11 wherein the first end body and/or the second end body include a registration tab, wherein the first expansion assembly and/or the second expansion assembly include a registration slot, and wherein the registration tab is positioned at least partially within the registration slot when the intervertebral spacer is in the expanded state.

13. The intervertebral spacer of example 12 wherein the registration slot is configured to at least partially prevent inward horizontal movement of one or more of the upper and lower supports when the registration tab is positioned at least partially within the registration slot.

14. The intervertebral spacer of any one of the examples 1-13 wherein the first and second expansion assembly are mechanically connected to cause the intervertebral spacer to expand sequentially in different directions.

15. The intervertebral spacer of any one of the examples 1-14 wherein the first locking feature is configured to clamp onto the locking region.

16. The intervertebral spacer of any one of the examples 1-15 wherein the first locking feature includes barbed locking arms.

17. A method for implanting an intervertebral spacer between first and second vertebral bodies of a subject's spine, the method comprising:
inserting the intervertebral spacer between the first and second vertebral bodies; and
transitioning the intervertebral spacer from an unexpanded state toward an expanded state, wherein transitioning the intervertebral spacer from the unexpanded state toward the expanded state includes—
   causing a first end body of the intervertebral spacer to move toward a second end body of the intervertebral spacer;
   causing a first expansion assembly of the intervertebral spacer and a second expansion assembly of the intervertebral spacer to move radially outward relative to a longitudinal axis of the intervertebral spacer; and
   causing a first locking feature of the first end body to engage a second locking feature of the second end body to at least partially prevent the intervertebral spacer from returning toward the first state.

18. The method of example 17 wherein causing the first expansion assembly of the intervertebral spacer and the second expansion assembly of the intervertebral spacer to move radially outward relative to the longitudinal axis includes causing the first expansion assembly and the second expansion assembly to move away from one another to horizontally expand the intervertebral spacer.

19. The method of any one of the examples 17-18 further comprising causing vertical expansion of the intervertebral spacer by:
causing a first upper support and a first lower support of the first expansion assembly to move away from one another; and
causing a second upper support and a second lower support of the second expansion assembly to move away from one another.

20. The method of any one of the examples 17-19 wherein causing vertical expansion of the intervertebral spacer includes causing the vertical expansion after causing horizontal expansion of the intervertebral spacer.

21. The method of any one of the examples 17-20 wherein causing the first locking feature of the first end body to engage the second locking feature of the second end body includes causing a ramped surface of the second end body to bend the first locking feature outwardly and away from the second end body.

22. The method of any one of the examples 17-21 wherein the first locking feature is a first upper locking feature, and wherein causing the first upper locking feature to engage the second locking feature includes causing at least a portion of the second end body to be positioned between the first upper locking feature and a first lower locking feature of the first end body.

23. The method of example 22 wherein the first upper locking feature and the first lower locking feature are positioned on opposite sides of the first end body.

24. A spinal system for treating a subject's spine, the spinal system comprising:
an intervertebral spacer configured to be positioned between first and second vertebral bodies of the subject's spine, the intervertebral spacer comprising—
   a first end body including a first locking feature; and
   a second end body including a second locking region configured to receive the first locking feature to move the intervertebral spacer from an unexpanded state to an expanded state and to cause the first locking feature to couple to the locking region to hold the intervertebral spacer in the expanded state; and
a delivery tool operably couplable to the intervertebral spacer and configured to transition the intervertebral spacer between the unexpanded state and the expanded state, the delivery tool comprising—
   a first engagement shaft configured to releasably couple the first end body of the intervertebral spacer to the delivery tool,
   a second engagement shaft configured to releasably couple the second end body of the intervertebral spacer to the delivery tool, the second engagement shaft including a notch, and
   an insertion limiter including a stop tab and transitionable between (i) a first position in which the stop tab contacts the notch to at least partially prevent further movement of the second engagement shaft toward the first locking feature of the intervertebral spacer, and (ii) a second position in which the stop tab is spaced apart from the notch to allow the second engagement shaft to move toward a first expansion locking feature.

25. The spinal system of any one of the examples 1-25 wherein in the expanded state, the movement of the second engagement shaft toward the first expansion locking feature uncouples the first locking feature from the second locking feature and allows the intervertebral spacer to be transitioned toward the first state.

26. The spinal system of any one of the examples 1-26 wherein the second engagement shaft is configured to bend the first locking feature away from the second locking feature to uncouple the first end body from the second end body.

27. The spinal system of any one of the examples 1-26 wherein the second end body includes a threaded attachment port configured to threadably receive threading of the second engagement shaft, wherein the threaded attachment port defines a gap such that a portion of the threading extends radially outwardly from the threaded attachment port, wherein the portion of the threading is configured to bend the first locking feature away from the second locking feature to uncouple the first end body from the second end body.

28. An intervertebral spacer, comprising:
a first expansion assembly configured to contact vertebral bodies of a subject and including a first upper support and a first lower support;
a second expansion assembly configured to contact the vertebral bodies and including a second upper support and a second lower support;
a first clamp linkage assembly rotatably coupled to the first expansion assembly and the second expansion assembly and including a pair of locking arms; and
a second linkage assembly rotatably coupled to the first expansion assembly and the second expansion assembly, wherein the second linkage assembly is configured push apart the pair of locking arms when the intervertebral spacer is moved toward an expanded state such that the pair of locking arms lock onto the second expansion assembly to hold the intervertebral spacer in the expanded state.

29. The intervertebral spacer of any one of the examples 1-28 wherein each of the pair of locking arms includes a hooked region positioned to contact the second linkage assembly when the intervertebral spacer is in the expanded state.

30. The intervertebral spacer of any one of the examples 1-29 wherein the second linkage assembly further includes ramped surfaces configured to deflect the pair of locking arms outwardly when the intervertebral spacer is moved toward the expanded state.

31. The intervertebral spacer of any one of the examples 1-30, wherein the first clamp linkage assembly includes in pair pivots.

32. The intervertebral spacer of any one of the examples 1-31, wherein the first and second expansion assemblies are mechanically connected to cause the intervertebral spacer to expand sequentially in different directions.

33. The intervertebral spacer of any one of the examples 1-32, wherein the locking arms are configured to be outwardly and then bias inwardly to clamp onto the second expansion assembly.

34. The intervertebral spacer of any one of the examples 1-33, wherein one or both locking arms include barbed end portions.

35. The intervertebral spacer of any one of the examples 1-34 wherein at least one of the locking arms includes a first locking feature includes a hooked region, wherein the second expansion assembly second includes a notch, and wherein the hooked region is positioned to contact the notch when the intervertebral spacer is in the expanded state.

36. The intervertebral spacer of any one of the examples 1-35 wherein the second linkage assembly includes a ramped surface configured to push apart the pair of locking arms.

37. The intervertebral spacer of any one of the examples 1-36 wherein the pair of locking arms are positioned on opposite sides of the second linkage assembly when the pair of locking arms hold the intervertebral spacer in the expanded state.

38. The intervertebral spacer of any one of the examples 1-37 wherein in the expanded state, the first upper support and the first lower support are spaced apart from one another, and the second upper support and the second lower support are spaced apart from one another.

39. The intervertebral spacer of any one of the examples 1-38, in the second state, the first upper support, the first lower support, the second upper support, and/or the second lower support are angled relative to a longitudinal axis of the intervertebral spacer.

40. The intervertebral spacer of any one of the examples 1-39 wherein, when the intervertebral spacer is in an unexpanded state, a first locking feature of the second linkage assembly is positioned at least partially between the first expansion assembly and the second expansion assembly.

41. The intervertebral spacer of example 40 wherein the second linkage assembly includes a recessed portion defining a locking feature gap in which at least a portion of one locking arms is positioned when the intervertebral spacer is in the unexpanded state.

42. The intervertebral spacer of any one of the examples 1-41, further comprising a plurality of links configured to couple the first and second expansion assemblies to first and second end bodies of the intervertebral spacer, wherein—
individual ones of the links include a rounded link head, and
the first upper support, the first lower support, the second upper support, and/or the second lower support include an expansion slot having a detent configured to receive the rounded link head when the intervertebral spacer is in the expanded state.

43. The intervertebral spacer of example 42 wherein at least one of the plurality of links includes a registration slot configured to mate with a corresponding link lock feature of the first or second end body to at least partially prevent rotational movement of the at least one link relative to the first or second end body.

44. The intervertebral spacer of any one of the examples 1-43, further including a registration tab, wherein the first expansion assembly and/or the second expansion assembly include a registration slot, and wherein the registration tab is positioned at least partially within the registration slot when the intervertebral spacer is in the expanded state.

45. The intervertebral spacer of example 44 wherein the registration slot is configured to at least partially prevent inward horizontal movement of one or more of the upper and lower supports when the registration tab is positioned at least partially within the registration slot.

### D. Conclusion

The disclosed medical devices, instruments, or any of their components can be made of a wide range of materials, including any biologically adaptable or compatible materials. Materials considered acceptable for biological implantation include, but are not limited to, stainless steel, titanium, tantalum, combination metallic alloys, various plastics, polymers, resins, ceramics, biologically absorbable materials and the like. Any assembly or its components can also be entirely or partially made of a shape memory material or other deformable material. It will be readily understood that the components of the invention, as generally described and illustrated in the Figures herein, could be arranged and designed in a wide variety of different configurations. Thus, the detailed description of the embodiments of the apparatus, system, and method, as represented in FIGS. 1 through 13D, is not intended to limit the scope of the invention, as claimed in this or any other application claiming priority to this application, but is merely representative of exemplary embodiments of the invention.

Devices, implants, instruments, methods, and related technologies are disclosed in U.S. Pat. Nos. 10,105,238; 10,898,340; 6,648,917; 6,562,074; 6,852,129; 6,863,673; 8,628,576; 9,308,099; 10,201,431; 10,945,859; 9,820,788; 10,105,238; U.S. application Ser. No. 16/565,403; U.S. application Ser. No. 16/687,520; U.S. application Ser. No. 17/125,633; U.S. application Ser. No. 15/793,950; U.S. application Ser. No. 16/394,244; U.S. application Ser. No. 15/970,212; U.S. application Ser. No. 15/500,969; U.S. application Ser. No. 18/335,737; U.S. application Ser. No. 18/464,949; U.S. application Ser. No. 18/470,140; U.S. application Ser. No. 18/073,364; U.S. Provisional App. No. 63/169,799; U.S. Provisional App. No. 63/163,489; U.S. Provisional App. No. 63/163,521; U.S. Provisional App. No. 63/169,804; U.S. Provisional App. No. 63/163,536; U.S. Provisional App. No. 61/442,482; U.S. Provisional App. No. 63/488,757, U.S. Provisional App. No. 63/611,913; U.S. Provisional App. No. 63/611,888; U.S. Provisional App. No. 63/504,248; U.S. Provisional App. No. 63/611,874; U.S. Provisional App. No. 63/550,554; PCT App. No. PCT/US20/49920; PCT App. No. PCT/US21/63881; PCT App. No. PCT/US22/19706, PCT App. No. PCT/US23/81937, and PCT App. No. PCT/US22/19706, the entireties of which are hereby incorporated by reference. These technologies can be used with, incorporated into, and/or combined with systems, methods, features, and components disclosed herein. For example, implants disclosed herein can have including features, such as locking screws, connectors, etc., disclosed in the incorporated by reference applications, publications, and patents. The implants can be utilized with the tool, instruments, guides (e.g., incision marking guides, placement guides, etc.), holders (e.g., multi-portal cannular holders, triangulation holders, instrument holders, pivoting instrument holders, etc.), delivery devices, or the like. All of the applications, publications, and patents cited herein are incorporated by reference in their entireties. Various features of the embodiments disclosed herein may be mixed and matched to provide additional configurations which fall within the scope of the invention. By way of non-limiting example, features and expansion capabilities of the embodiments disclosed herein may be combined to provide a symmetrical spacer embodiment providing no lordotic correction; a symmetrical spacer embodiment which provides a lordotic correction; an asymmetrical spacer embodiment providing no lordotic correction; and an asymmetrical spacer embodiment which provides a lordotic correction. One or more embodiments may be implanted together to provide the precise support and/or correction needed to restore sagittal alignment and balance.

The phrases “connected to,” “coupled to,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The terms “upper” and “lower,” “top” and “bottom,” “front” and “back” are used as relative terms herein for ease of description and understanding. It is understood that in embodiments of the disclosure, upper and lower, top and bottom, and/or front and back entities may be reversed.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. To the extent any material incorporated herein by reference conflicts with the present disclosure, the present disclosure controls.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the invention.

While specific embodiments and applications of the present invention have been illustrated and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention.

What is claimed is:

1. An intervertebral spacer, comprising:
a first expansion assembly including a first upper support and a first lower support;
a second expansion assembly including a second upper support and a second lower support;
a first end body coupled to the first expansion assembly and the second expansion assembly, the first end body including a first locking feature; and
a second end body coupled to the first expansion assembly and the second expansion assembly, the second end body including a locking region configured to receive the first locking feature when driving apart and expanding the first and second expansion assemblies to move the intervertebral spacer from an unexpanded state to an expanded state, and to cause the first locking feature to couple to the locking region to hold the intervertebral spacer in the expanded state.

2. The intervertebral spacer of claim 1 wherein the first locking feature includes a hooked region, wherein the second locking feature includes a notch, and wherein the hooked region is positioned to contact the notch when the intervertebral spacer is in the expanded state.

3. The intervertebral spacer of claim 1 wherein the second end body further includes a ramped surface configured to deflect the first locking feature outwardly from the second end body when the intervertebral spacer is moved from the unexpanded state to the expanded state.

4. The intervertebral spacer of claim 1 wherein the first locking feature is a first upper locking feature configured to couple to a first side of the second end body, the first end body further comprising a first lower locking feature configured to couple to a second side of the second end body opposite the first side when the intervertebral spacer is in the expanded state.

5. The intervertebral spacer of claim 1 wherein:
in the unexpanded state—
the first expansion assembly and the second expansion assembly are positioned at least partially between the first end body and the second end body, and
the first locking feature is spaced apart from the second locking feature, and
in the expanded state—
the first end body and the second end body are positioned at least partially between the first expansion assembly and the second expansion assembly, and
the first locking feature is coupled to the second locking feature to at least partially prevent the intervertebral spacer from returning to the unexpanded state.

6. The intervertebral spacer of claim 1 wherein in the expanded state, the first upper support and the first lower support are spaced apart from one another, and the second upper support and the second lower support are spaced apart from one another.

7. The intervertebral spacer of claim 1 wherein, in the second state, the first upper support, the first lower support, the second upper support, and/or the second lower support are angled relative to a longitudinal axis of the intervertebral spacer.

8. The intervertebral spacer of claim 1 wherein, in the unexpanded state, the first locking feature is positioned at least partially between the first expansion assembly and the second expansion assembly.

9. The intervertebral spacer of claim 8 wherein the first upper support, the first lower support, the second upper support, and/or the second lower support include a recessed portion defining a locking feature gap in which at least a portion of the first locking feature is positioned when the intervertebral spacer is in the unexpanded state.

10. The intervertebral spacer of claim 1, further comprising a plurality of links configured to couple the first and second expansion assemblies to the first and second end bodies, wherein—
individual ones of the links include a rounded link head, and
the first upper support, the first lower support, the second upper support, and/or the second lower support include an expansion slot having a detent configured to receive the rounded link head when the intervertebral spacer is in the expanded state.

11. The intervertebral spacer of claim 10 wherein at least one of the plurality of links includes a registration slot configured to mate with a corresponding link lock feature of the first or second end body to at least partially prevent rotational movement of the at least one link relative to the first or second end body.

12. The intervertebral spacer of claim 1 wherein the first end body and/or the second end body include a registration tab, wherein the first expansion assembly and/or the second expansion assembly include a registration slot, and wherein the registration tab is positioned at least partially within the registration slot when the intervertebral spacer is in the expanded state.

13. The intervertebral spacer of claim 12 wherein the registration slot is configured to at least partially prevent inward horizontal movement of one or more of the upper and lower supports when the registration tab is positioned at least partially within the registration slot.

14. The intervertebral spacer of claim 1, wherein the first and second expansion assembly are mechanically connected to cause the intervertebral spacer to expand sequentially in different directions.

15. The intervertebral spacer of claim 1, wherein the first locking feature is configured to clamp onto the locking region.

16. The intervertebral spacer of claim 1, wherein the first locking feature includes barbed locking arms.

17. A method for implanting an intervertebral spacer between first and second vertebral bodies of a subject's spine, the method comprising:
inserting the intervertebral spacer between the first and second vertebral bodies; and
transitioning the intervertebral spacer from an unexpanded state toward an expanded state, wherein transitioning the intervertebral spacer from the unexpanded state toward the expanded state includes—
causing a first end body of the intervertebral spacer to move toward a second end body of the intervertebral spacer;
causing a first expansion assembly of the intervertebral spacer and a second expansion assembly of the intervertebral spacer to move radially outward relative to a longitudinal axis of the intervertebral spacer; and
causing a first locking feature of the first end body to engage a second locking feature of the second end body to at least partially prevent the intervertebral spacer from returning toward the first state.

18. The method of claim 17 wherein causing the first expansion assembly of the intervertebral spacer and the second expansion assembly of the intervertebral spacer to move radially outward relative to the longitudinal axis includes causing the first expansion assembly and the second expansion assembly to move away from one another to horizontally expand the intervertebral spacer.

19. The method of claim 17 further comprising causing vertical expansion of the intervertebral spacer by:
causing a first upper support and a first lower support of the first expansion assembly to move away from one another; and
causing a second upper support and a second lower support of the second expansion assembly to move away from one another.

20. The method of claim 19 wherein causing vertical expansion of the intervertebral spacer includes causing the vertical expansion after causing horizontal expansion of the intervertebral spacer.

21. The method of claim 17 wherein causing the first locking feature of the first end body to engage the second locking feature of the second end body includes causing a ramped surface of the second end body to bend the first locking feature outwardly and away from the second end body.

22. The method of claim 17 wherein the first locking feature is a first upper locking feature, and wherein causing the first upper locking feature to engage the second locking feature includes causing at least a portion of the second end body to be positioned between the first upper locking feature and a first lower locking feature of the first end body.

23. The method of claim 22 wherein the first upper locking feature and the first lower locking feature are positioned on opposite sides of the first end body.

24. A spinal system for treating a subject's spine, the spinal system comprising:
an intervertebral spacer configured to be positioned between first and second vertebral bodies of the subject's spine, the intervertebral spacer comprising—
a first end body including a first locking feature; and
a second end body including a second locking region configured to receive the first locking feature to move the intervertebral spacer from an unexpanded state to an expanded state and to cause the first locking feature to couple to the locking region to hold the intervertebral spacer in the expanded state; and
a delivery tool operably couplable to the intervertebral spacer and configured to transition the intervertebral spacer between the unexpanded state and the expanded state, the delivery tool comprising—
a first engagement shaft configured to releasably couple the first end body of the intervertebral spacer to the delivery tool,
a second engagement shaft configured to releasably couple the second end body of the intervertebral spacer to the delivery tool, the second engagement shaft including a notch, and
an insertion limiter including a stop tab and transitionable between (i) a first position in which the stop tab contacts the notch to at least partially prevent further movement of the second engagement shaft toward the first locking feature of the intervertebral spacer, and (ii) a second position in which the stop tab is spaced apart from the notch to allow the second engagement shaft to move toward a first expansion locking feature.

25. The spinal system of claim 24 wherein in the expanded state, the movement of the second engagement shaft toward the first expansion locking feature uncouples the first locking feature from the second locking feature and allows the intervertebral spacer to be transitioned toward the first state.

26. The spinal system of claim 24 wherein the second engagement shaft is configured to bend the first locking feature away from the second locking feature to uncouple the first end body from the second end body.

27. The spinal system of claim 26 wherein the second end body includes a threaded attachment port configured to threadably receive threading of the second engagement shaft, wherein the threaded attachment port defines a gap such that a portion of the threading extends radially outwardly from the threaded attachment port, wherein the portion of the threading is configured to bend the first locking feature away from the second locking feature to uncouple the first end body from the second end body.

28. An intervertebral spacer, comprising:
a first expansion assembly configured to contact vertebral bodies of a subject and including a first upper support and a first lower support;
a second expansion assembly configured to contact the vertebral bodies and including a second upper support and a second lower support;
a first clamp linkage assembly rotatably coupled to the first expansion assembly and the second expansion assembly and including a pair of locking arms; and
a second linkage assembly rotatably coupled to the first expansion assembly and the second expansion assembly, wherein the second linkage assembly is configured push apart the pair of locking arms when the intervertebral spacer is moved toward an expanded state such that the pair of locking arms lock onto the second expansion assembly to hold the intervertebral spacer in the expanded state.

29. The intervertebral spacer of claim 28 wherein each of the pair of locking arms includes a hooked region positioned to contact the second linkage assembly when the intervertebral spacer is in the expanded state.

30. The intervertebral spacer of claim 28 wherein the second linkage assembly further includes ramped surfaces configured to deflect the pair of locking arms outwardly when the intervertebral spacer is moved toward the expanded state.

31. The intervertebral spacer of claim 28, wherein the first clamp linkage assembly includes in pair pivots.

32. The intervertebral spacer of claim 28, wherein the first and second expansion assemblies are mechanically connected to cause the intervertebral spacer to expand sequentially in different directions.

33. The intervertebral spacer of claim 28, wherein the locking arms are configured to be outwardly and then bias inwardly to clamp onto the second expansion assembly.

34. The intervertebral spacer of claim 28, wherein one or both locking arms include barbed end portions.

35. The intervertebral spacer of claim 28 wherein at least one of the locking arms includes a first locking feature includes a hooked region, wherein the second expansion assembly second includes a notch, and wherein the hooked region is positioned to contact the notch when the intervertebral spacer is in the expanded state.

36. The intervertebral spacer of claim 28 wherein the second linkage assembly includes a ramped surface configured to push apart the pair of locking arms.

37. The intervertebral spacer of claim 28 wherein the pair of locking arms are positioned on opposite sides of the second linkage assembly when the pair of locking arms hold the intervertebral spacer in the expanded state.

38. The intervertebral spacer of claim 28 wherein in the expanded state, the first upper support and the first lower support are spaced apart from one another, and the second upper support and the second lower support are spaced apart from one another.

39. The intervertebral spacer of claim 28, wherein, in the second state, the first upper support, the first lower support, the second upper support, and/or the second lower support are angled relative to a longitudinal axis of the intervertebral spacer.

40. The intervertebral spacer of claim 28 wherein, when the intervertebral spacer is in an unexpanded state, a first locking feature of the second linkage assembly is positioned at least partially between the first expansion assembly and the second expansion assembly.

41. The intervertebral spacer of claim 40 wherein the second linkage assembly includes a recessed portion defining a locking feature gap in which at least a portion of one locking arms is positioned when the intervertebral spacer is in the unexpanded state.

42. The intervertebral spacer of claim 28, further comprising a plurality of links configured to couple the first and second expansion assemblies to first and second end bodies of the intervertebral spacer, wherein—
individual ones of the links include a rounded link head, and
the first upper support, the first lower support, the second upper support, and/or the second lower support include an expansion slot having a detent configured to receive the rounded link head when the intervertebral spacer is in the expanded state.

43. The intervertebral spacer of claim 42 wherein at least one of the plurality of links includes a registration slot configured to mate with a corresponding link lock feature of the first or second end body to at least partially prevent rotational movement of the at least one link relative to the first or second end body.

44. The intervertebral spacer of claim 28, further including a registration tab, wherein the first expansion assembly and/or the second expansion assembly include a registration slot, and wherein the registration tab is positioned at least partially within the registration slot when the intervertebral spacer is in the expanded state.

45. The intervertebral spacer of claim 44 wherein the registration slot is configured to at least partially prevent inward horizontal movement of one or more of the upper and lower supports when the registration tab is positioned at least partially within the registration slot.
